(19) United States
(12) Patent Application Publication
(10) Pub. No.: US 2024/0215438 A1
(43) Pub. Date: Jun. 27, 2024

(54) ORGANIC LIGAND INCLUDING AZIDE GROUP, AND ORGANIC SEMICONDUCTOR THIN FILM AND OPTOELECTRONIC DEVICE INCLUDING THE ORGANIC LIGAND

(71) Applicants: Samsung Display Co., Ltd., Yongin-si (KR); POSTECH Research and Business Development Foundation, Pohang-si (KR)

(72) Inventors: Kunwook Cho, Yongin-si (KR); Hwasook Ryu, Yongin-si (KR); Syed Zahid Hassan, Pohang-si (KR); Seokgyu Yoon, Yongin-si (KR); Juhyeok Lee, Pohang-si (KR); Daesung Chung, Pohang-si (KR); Kyumin Sim, Pohang-si (KR)

(73) Assignees: Samsung Display Co., Ltd., Yongin-si (KR); POSTECH Research and Business Development Foundation, Pohang-si (KR)

(21) Appl. No.: 18/494,858

(22) Filed: Oct. 26, 2023

(30) Foreign Application Priority Data
Dec. 9, 2022 (KR) 10-2022-0171858

(51) Int. Cl. H10K 85/60; C07C 243/00; C07D 213/79; C07D 239/28; C07D 239/42; C07D 307/68; C09K 11/06

(52) U.S. Cl. CPC H10K 85/60; C07C 243/00; C07D 213/79; C07D 239/28; C07D 239/42; C07D 307/68; C09K 11/06; H10K 85/653; H10K 85/654; H10K 50/16

(57) ABSTRACT

Embodiments provide an organic compound, an organic ligand, an optoelectronic device including the organic compound or the organic ligand, an electronic apparatus including the optoelectronic device, an electronic equipment including the electronic apparatus, and an organic semiconductor thin film including the organic compound or the organic ligand. The optoelectronic device includes a first electrode, a second electrode facing the first electrode, an interlayer between the first electrode and the second electrode, and an organic compound represented by Formula 1 or an organic ligand represented by Formula 1A or Formula 1B, wherein Formulae 1, 1A, and 1B are explained in the specification:

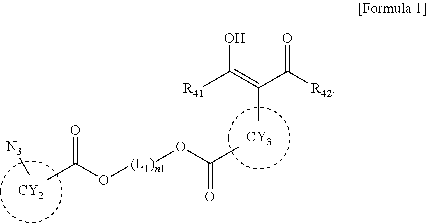

### CROSS-REFERENCE TO RELATED APPLICATION(S)

This application claims priority to and benefits of Korean Patent Application No. 10-2022-0171858 under 35 U.S.C. § 119, filed on Dec. 9, 2022, in the Korean Intellectual Property Office, the entire contents of which are incorporated herein by reference.

### BACKGROUND

### 1. Technical Field

Embodiments relate to an organic ligand including an azide group, and an organic semiconductor thin film and an optoelectronic device that include the organic ligand.

### 2. Description of the Related Art

Optoelectronic devices are devices that convert optical energy or optical signals into electrical energy or electrical signals. Examples of an optoelectronic device are an optical or solar cell, which converts optical energy into electrical energy, an optical detector or sensor, which detects optical energy and converts it into electrical signals, and the like.

Organic semiconductors have attracted attention throughout the industry due to the various advantages thereof, such as low process costs through solution processing, mass production, flexibility, light weight, and ease of optoelectronic characteristic control through molecular structure control. Such organic semiconductors may be used in optoelectronic devices.

Organic semiconductors having inverted structures using oxide semiconductors exhibit high stability, and thus have attracted great attention. In the case of an inverted structure, an interlayer having characteristics such as transparency, high electron mobility, and environment-friendly processing is required, and oxide semiconductors satisfy all of such requirements.

However, in the case of an oxide semiconductor, low optical reliability is considered an issue to be overcome. The low optical reliability is due to defects related to oxygen inside the oxide semiconductor. For example, when light is applied, an oxygen vacancy acts as a thin electron donor defect, thereby reducing the performance of a device. Such an issue appears more severely in the case of an oxide semiconductor manufactured by a low-temperature solution process.

It is to be understood that this background of the technology section is, in part, intended to provide useful background for understanding the technology. However, this background of the technology section may also include ideas, concepts, or recognitions that were not part of what was known or appreciated by those skilled in the pertinent art prior to a corresponding effective filing date of the subject matter disclosed herein.

### SUMMARY

Embodiments include an organic compound or an organic ligand including an azide group, and an organic semiconductor thin film having increased crystallinity and reduced defects by including the organic compound or the organic ligand. Another embodiment includes an optoelectronic device having a high photocurrent value by including the organic semiconductor thin film.

Additional aspects will be set forth in part in the description which follows and, in part, will be apparent from the description, or may be learned by practice of the embodiments.

According to embodiments, an optoelectronic device may include a first electrode, a second electrode facing the first electrode, an interlayer between the first electrode and the second electrode, and an organic compound represented by Formula 1 or an organic ligand represented by Formula 1A or Formula 1B:

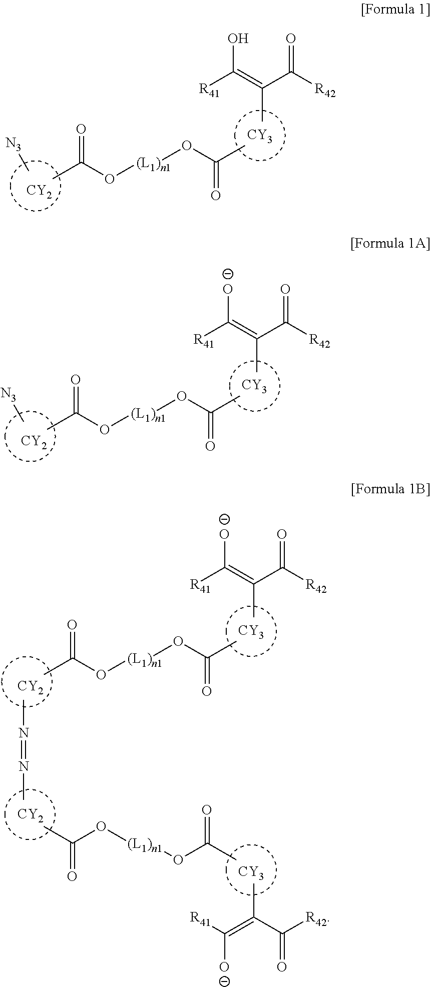

In Formulae 1, 1A, and 1B,
ring CY₂ may be a C₃-C₆₀ carbocyclic group or a C₁-C₆₀ heterocyclic group, each unsubstituted or substituted with at least one R₂,
ring CY₃ may be a C₃-C₆₀ carbocyclic group or a C₁-C₆₀ heterocyclic group, each unsubstituted or substituted with at least one R₃,
L₁ may be: a single bond; *—O—*′, *—S—*′, or *—Se—*′; or a C₁-C₆₀ alkylene group, a C₂-C₆₀ alkenylene group, a C₂-C₆₀ alkynylene group, a C₆-C₆₀ arylene group, or a C₁-C₆₀ heteroarylene group, each unsubstituted or substituted with at least one R₁,
n1 may be an integer from 0 to 10,
when n1 is 0, L₁ may be a single bond,
when n1 is 2 or more, a plurality of L₁ may be identical to or different from each other,
R₁, R₂, and R₃ may each independently be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₃-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
R₄₁ and R₄₂ may each independently be:
hydrogen, deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁)(Q₁₂), —B(Q₁₁)(Q₁₂), —C(═O)(Q₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₂-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

In an embodiment, the interlayer may include a hole transport region, a photoactive layer, an electron transport region, or any combination thereof; and the organic compound or the organic ligand may be included in the electron transport region.

In an embodiment, the optoelectronic device may include two or more organic compounds each independently represented by Formula 1; and an azide group of one of the two or more organic compounds may form at least one cross-link with an azide group of another of the two or more organic compounds.

In an embodiment, the optoelectronic device may further include metal oxide particles, wherein the metal oxide particles may form at least one coordinate bond with the organic compound or the organic ligand.

In an embodiment, the interlayer may include a hole transport region, a photoactive layer, an electron transport region, or any combination thereof; and the organic compound or the organic ligand, and the metal oxide particles may be included in the electron transport region.

In an embodiment, the metal oxide particles may be ZnO, ZrO₂, TiO₂, Nb₂O₅, Al₂O₃, SnO₂, or AZO.

According to embodiments, an electronic apparatus may include the optoelectronic device.

In an embodiment, the electronic apparatus may further include: a thin-film transistor electrically connected to the first electrode of the optoelectronic device; an emission layer between the first electrode and the second electrode of the optoelectronic device; and a color filter, a color conversion layer, a touch screen layer, a polarizing layer, or any combination thereof.

According to embodiments, an electronic equipment may include the electronic apparatus, wherein the electronic equipment may be a flat panel display, a curved display, a computer monitor, a medical monitor, a television, an advertisement board, an indoor light, an outdoor light, a signaling light, a head-up display, a fully transparent display, a partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a microdisplay, a three-dimensional (3D) display, a virtual reality display, an augmented reality display, a vehicle, a video wall including multiple displays tiled together, a theater screen, a stadium screen, a phototherapy device, or a sign.

According to embodiments, an organic compound may be represented by Formula 1, which is explained herein.

In an embodiment, ring CY₂ may be a C₆-C₃₀ aryl group or a C₁-C₃₀ heteroaryl group, each substituted with at least one R₂; and ring CY₃ may be a C₆-C₃ aryl group or a C₁-C₃₀ heteroaryl group, each substituted with at least one R₃.

In an embodiment, ring CY₂ may be a moiety represented by one of Formulae 2-1 to 2-6, which are explained below.

In an embodiment, ring CY₃ is a moiety represented by one of Formulae 3-1 to 3-6, which are explained below.

In an embodiment, L₁ may be *—O—*′, —S—*′*—C(R₁₁)(R₁₂)—*′, *—C(R₁₁)═(R₁₂)—*′, *—C≡C—*, or a phenylene group unsubstituted or substituted with at least one R₁,
R₁₁ and R₁₂ may each independently be: hydrogen; or as defined in connection with R₁ in Formula 1,
R₁ is as described in Formula 1, and
and *′ each indicate a binding site to a neighboring atom.

In an embodiment, R₁, R₂, and R₃ may each independently be:
deuterium, —F, —Cl, —Br, —I, a cyano group, or a nitro group; or
a C₁-C₂₀ alkyl group, a C₂-C₂₀ alkenyl group, a C₂-C₂₀ alkynyl group, or a C₂-C₂₀ alkoxy group, each unsubstituted or substituted with deuterium, —F, —Cl, —Br, —I, a cyano group, a nitro group, Si(Q₁₁)(Q₁₂)(Q₁₃), —B(Q₁₁)(Q₁₂), —C(═O)(Q₁₁), —S(═O)₂(Q₁₁), —P(═O)(Q₁₁)(Q₁₂), or any combination thereof.

In an embodiment, the organic compound may be represented by one of Formulae 1-1 to 1-3, which are explained below.

In an embodiment, the organic compound may be one of Compounds 1, 2, and 4 to 25, which are explained below.

According to embodiments, an organic semiconductor thin film may include metal oxide particles, and an organic compound represented by Formula 1 or an organic ligand represented by Formula 1A or Formula 1B, which are explained herein.

In an embodiment, the metal oxide particles may form at least one coordinate bond with the organic compound or the organic ligand.

In an embodiment, the organic semiconductor thin film may include two or more organic compounds each independently represented by Formula 1; and an azide group of one of the two or more organic compounds may form at least one cross-link with an azide group of another of the two or more organic compounds.

It is to be understood that the embodiments above are described in a generic and explanatory sense only and not for the purpose of limitation, and the disclosure is not limited to the embodiments described above.

### BRIEF DESCRIPTION OF THE DRAWINGS

The above and other aspects and features of the disclosure will be more apparent by describing in detail embodiments thereof with reference to the accompanying drawings, in which:

FIG. 1 is a schematic cross-sectional view of an optoelectronic device according to an embodiment;

FIG. 2 is a schematic cross-sectional view of an optoelectronic device according to an embodiment;

FIG. 3 is a graph showing external quantum efficiency (EQE) versus wavelength for the optoelectronic devices according to Example 2 and Comparative Example 1;

FIG. 4 is a graph showing dark current density verses voltage (J-V curve) of the optoelectronic devices according to Example 2 and Comparative Example 1;

FIG. 5 is a schematic cross-sectional view of an electronic apparatus according to an embodiment;

FIG. 6 is a schematic cross-sectional view of an electronic apparatus according to another embodiment;

FIG. 7 is a schematic perspective view of electronic equipment including an optoelectronic device according to an embodiment;

FIG. 8 is a schematic view of the exterior of a vehicle as an electronic equipment including an optoelectronic device according to an embodiment; and

FIGS. 9A to 9C are each a schematic view of the interior of a vehicle, according to an embodiment.

### DETAILED DESCRIPTION OF THE EMBODIMENTS

The disclosure will now be described more fully hereinafter with reference to the accompanying drawings, in which embodiments are shown. This disclosure may, however, be embodied in different forms and should not be construed as limited to the embodiments set forth herein. Rather, these embodiments are provided so that this disclosure will be thorough and complete, and will fully convey the scope of the disclosure to those skilled in the art.

In the drawings, the sizes, thicknesses, ratios, and dimensions of the elements may be exaggerated for ease of description and for clarity. Like numbers refer to like elements throughout.

In the description, it will be understood that when an element (or region, layer, part, etc.) is referred to as being “on”, “connected to”, or “coupled to” another element, it can be directly on, connected to, or coupled to the other element, or one or more intervening elements may be present therebetween. In a similar sense, when an element (or region, layer, part, etc.) is described as “covering” another element, it can directly cover the other element, or one or more intervening elements may be present therebetween.

In the description, when an element is “directly on,” “directly connected to,” or “directly coupled to” another element, there are no intervening elements present. For example, “directly on” may mean that two layers or two elements are disposed without an additional element such as an adhesion element therebetween.

As used herein, the expressions used in the singular such as “a,” “an,” and “the,” are intended to include the plural forms as well, unless the context clearly indicates otherwise.

As used herein, the term “and/or” includes any and all combinations of one or more of the associated listed items. For example, “A and/or B” may be understood to mean “A, B, or A and B.” The terms “and” and “or” may be used in the conjunctive or disjunctive sense and may be understood to be equivalent to “and/or”.

In the specification and the claims, the term “at least one of” is intended to include the meaning of “at least one selected from the group consisting of” for the purpose of its meaning and interpretation. For example, “at least one of A, B, and C” may be understood to mean A only, B only, C only, or any combination of two or more of A, B, and C, such as ABC, ACC, BC, or CC. When preceding a list of elements, the term, “at least one of,” modifies the entire list of elements and does not modify the individual elements of the list.

It will be understood that, although the terms first, second, etc. may be used herein to describe various elements, these elements should not be limited by these terms. These terms are only used to distinguish one element from another element.

Thus, a first element could be termed a second element without departing from the teachings of the disclosure. Similarly, a second element could be termed a first element, without departing from the scope of the disclosure.

The spatially relative terms “below”, “beneath”, “lower”, “above”, “upper”, or the like, may be used herein for ease of description to describe the relations between one element or component and another element or component as illustrated in the drawings. It will be understood that the spatially relative terms are intended to encompass different orientations of the device in use or operation, in addition to the orientation depicted in the drawings. For example, in the case where a device illustrated in the drawing is turned over, the device positioned “below” or “beneath” another device may be placed “above” another device. Accordingly, the illustrative term “below” may include both the lower and upper positions. The device may also be oriented in other directions and thus the spatially relative terms may be interpreted differently depending on the orientations.

The terms “about” or “approximately” as used herein is inclusive of the stated value and means within an acceptable range of deviation for the recited value as determined by one of ordinary skill in the art, considering the measurement in question and the error associated with measurement of the recited quantity (i.e., the limitations of the measurement system). For example, “about” may mean within one or more standard deviations, or within ±20%, ±10%, or ±5% of the stated value.

It should be understood that the terms “comprises,” “comprising,” “includes,” “including,” “have,” “having,” “contains,” “containing,” and the like are intended to specify the presence of stated features, integers, steps, operations, elements, components, or combinations thereof in the disclosure, but do not preclude the presence or addition of one or more other features, integers, steps, operations, elements, components, or combinations thereof.

Unless otherwise defined or implied herein, all terms (including technical and scientific terms) used have the same meaning as commonly understood by those skilled in the art to which this disclosure pertains. It will be further understood that terms, such as those defined in commonly used dictionaries, should be interpreted as having a meaning that is consistent with their meaning in the context of the relevant art and should not be interpreted in an ideal or excessively formal sense unless clearly defined in the specification.

An embodiment provides an optoelectronic device which may include: a first electrode; a second electrode facing the first electrode; an interlayer arranged between the first electrode and the second electrode; and an organic compound represented by Formula 1 or an organic ligand represented by Formula 1A or 1B.

The organic compound or organic ligand will be described.

The organic compound may be represented by Formula 1, and the organic ligand may be represented by Formula 1A or Formula 1B:

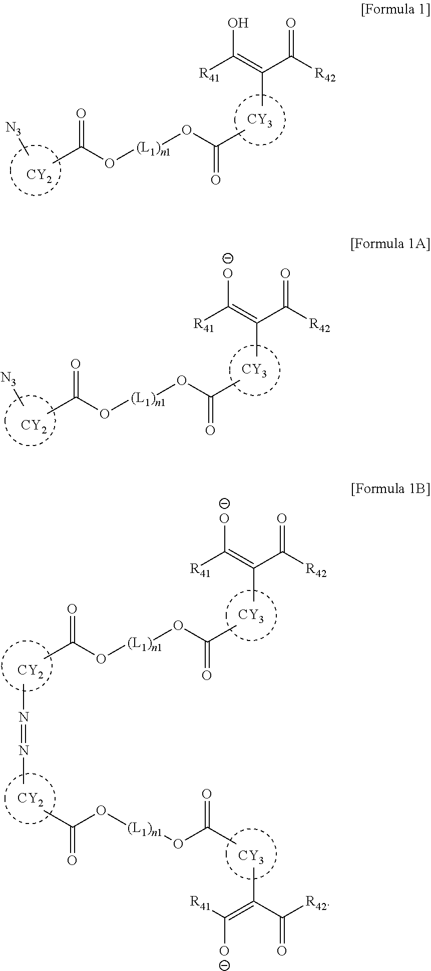

In Formulae 1, 1A, and 1B,
ring CY₂ may be a C₃-C₆₀ carbocyclic group or a C₁-C₆₀ heterocyclic group, each unsubstituted or substituted with at least one R₂,
ring CY₃ may be a C₃-C₆₀ carbocyclic group or a C₁-C₆₀ heterocyclic group, each unsubstituted or substituted with at least one R₃,
L₁ may be: a single bond; *—O—*′; *—S—*′; or *—Se—*′; or a C₁-C₆₀ alkylene group, a C₂-C₆₀ alkenylene group, a C₂-C₆₀ alkynylene group, a C₆-C₆₀ arylene group, or a C₁-C₆₀ heteroarylene group, each unsubstituted or substituted with at least one R₁,
n1 may be an integer from 0 to 10,
when n1 is 0, L₁ may be a single bond,
when n1 is 2 or more, a plurality of L₁ may be identical to or different from each other,
R₁, R₂, and R₃ may each independently be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
R₄₁ and R₄₂ may each independently be:
hydrogen, deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₃-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂,)(Q₂₂), or any combination thereof; or
—Si(Q₃,)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

In an embodiment, ring CY₂ may be a C₆-C₃₀ aryl group or a C₁-C₃ heteroaryl group, each substituted with at least one R₂, and ring CY₃ may be a C₆-C₃ aryl group or a C₁-C₃ heteroaryl group, each substituted with at least one R₃.

In an embodiment, ring CY₂ may be a moiety represented by one of Formulae 2-1 to 2-6:

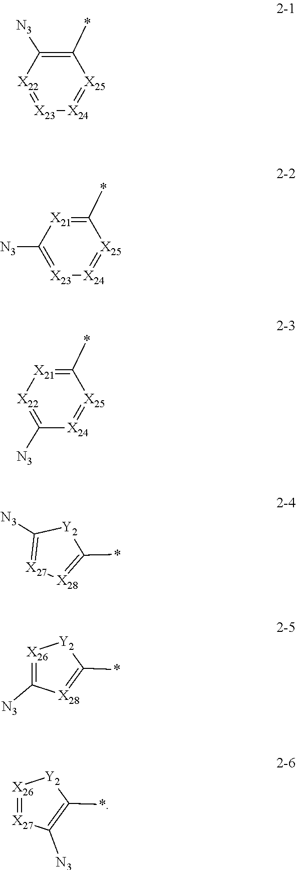

In Formulae 2-1 to 2-6,
X₂₁ may be N or C(R₂₁), X₂₂ may be N or C(R₂₂), X₂₃ may be N or C(R₂₃), X₂₄ may be N or C(R₂₄), X₂₅ may be N or C(R₂₅), X₂₆ may be N or C(R₂₆), X₂₇ may be N or C(R₂₇), and X₂₈ may be N or C(R₂₈),
R₂₁ to R₂₈ may each independently be: hydrogen; or as defined in connection with R₂ in Formula 1,
Y₂ may be O, S, or Se, and
* may indicate a binding site to a neighboring atom.

In an embodiment, ring CY₂ may be a moiety represented by one of Formulae 2A to 2M:

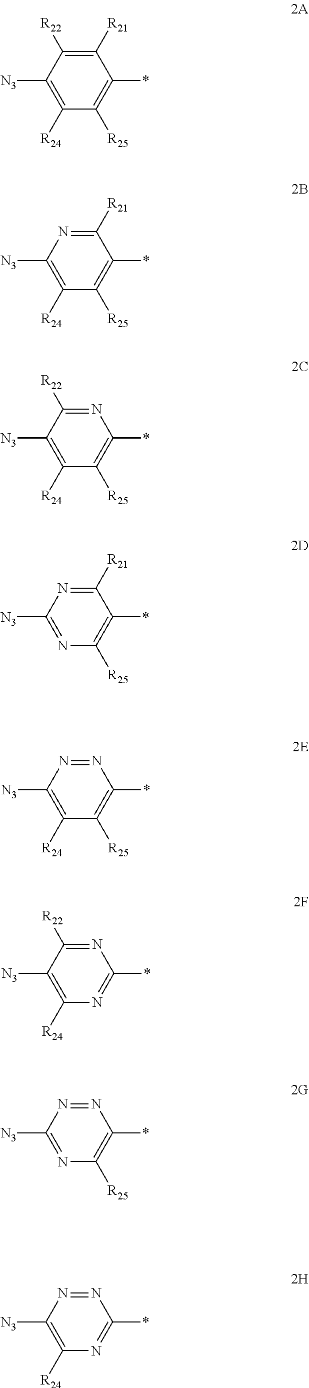

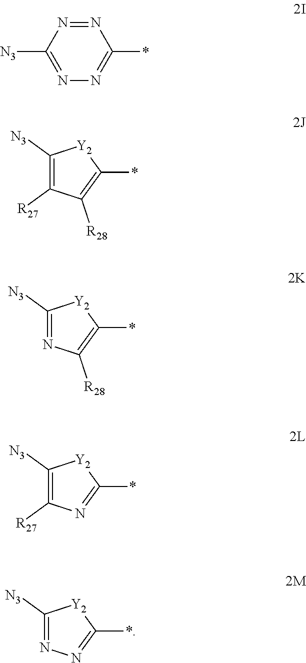

In Formulae 2A to 2M,
R₂₁ to R₂₅, R₂₇, and R₂₈ may each independently be: hydrogen; or as defined in connection with R₂ in Formula 1,
Y₂ may be O, S, or Se, and
* may indicate a binding site to a neighboring atom.

In an embodiment, ring CY₃ may be a moiety represented by one of Formulae

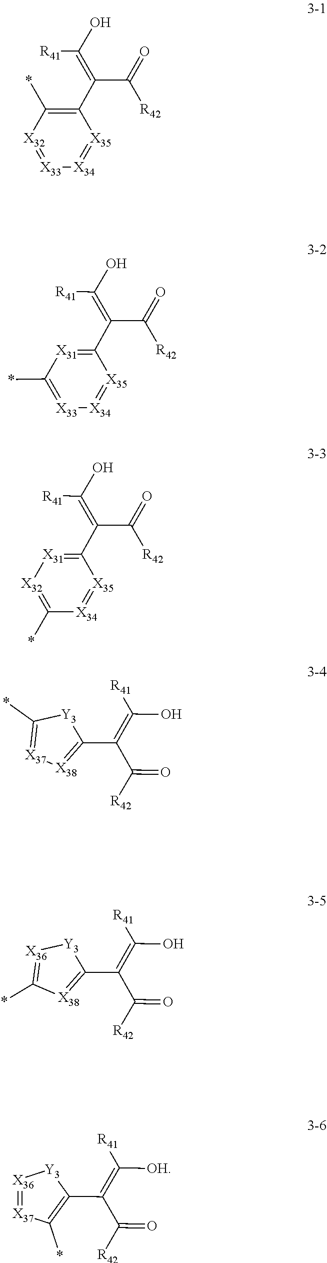

In Formulae 3-1 to 3-6,
X₃₁ may be N or C(R₃₁), X₃₂ may be N or C(R₃₂), X₃a may be N or C(R₃₃), X₃₄ may be N or C(R₃₄), X₃₅ may be N or C(R₃₅), X₃₆ may be N or C(R₃₆), X₃₇ may be N or C(R₃₇), and X₃₈ may be N or C(R₃₈),
R₃₁ to R₃₈ may each independently be: hydrogen; or as defined in connection with R₃ in Formula 1,
R₄₁ and R₄₂ are as defined in Formula 1,
Y₃ may be O, S, or Se, and
* may indicate a binding site to a neighboring atom.

In an embodiment, ring CY₃ may be a moiety represented by one of Formulae 3A to 3M:

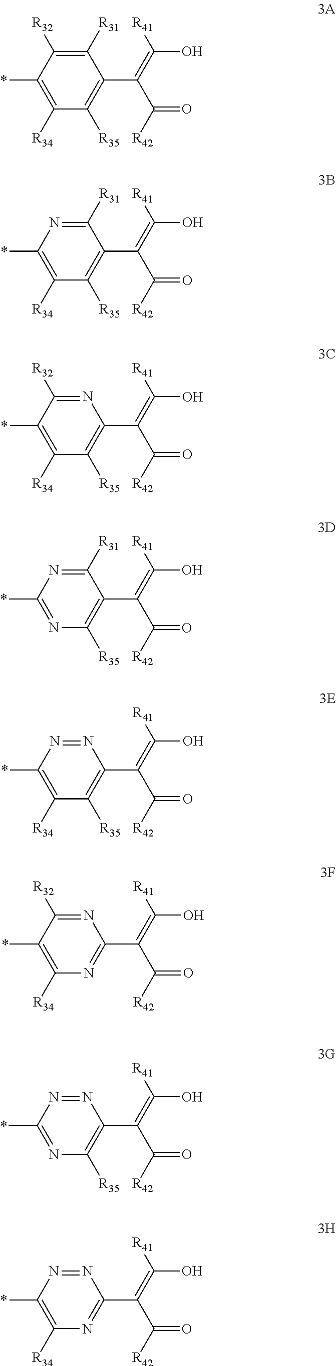

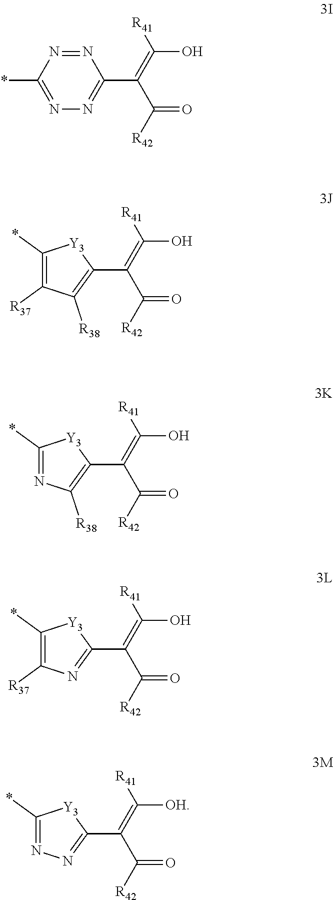

In Formulae 3A to 3M,
R₃₁ to R₃₅, R₃₇, and R₃₈ may each independently be: hydrogen; or as defined in connection with R₃ in Formula 1,
R₄₁ and R₄₂ are as defined in Formula 1,
Y₃ may be O, S, or Se, and
* may indicate a binding site to a neighboring atom.

In an embodiment, L₁ may be *—O—*, *—S—,*′ *—C(R₁₁)(R₁₂)—*′, *—C(R₁₁)═(R₁₂)—*′, *—C≡C—*′, or a phenylene group unsubstituted or substituted with at least one R₁,
R₁₁ and R₁₂ may each independently be: hydrogen; or as defined in connection with R₁ in Formula 1,
R₁ is as described in Formula 1, and * and *′ may each indicate a binding site to a neighboring atom.

In an embodiment, L₁ may be *—O—*, *—C(R₁₁)(R₁₂)—*′, or a substituent represented by one of Formulae 1A to 1C:

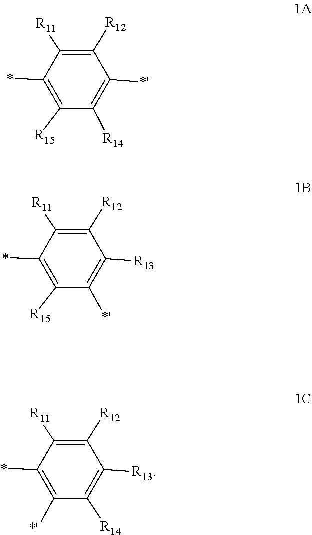

In Formulae 1A to 1C,
R₁₁ to R₁₅ may each independently be: hydrogen; or as defined in connection with R₁ in Formula 1, and * and *′ may each indicate a binding site to a neighboring atom.

In an embodiment, R₁, R₂, and R₃ may each independently be:
deuterium, —F, —Cl, —Br, —I, a cyano group, or a nitro group; or
a C₁-C₂₀ alkyl group, a C₂-C₂₀ alkenyl group, a C₂-C₂₀ alkynyl group, or a C₂-C₂₀ alkoxy group, each unsubstituted or substituted with deuterium, —F, —Cl, —Br, —I, a cyano group, a nitro group, Si(Q₁₁)(Q₁₂)(Q₁₃), —B(Q₁₁)(Q₁₂), —C(═O)(Q₁₁), —S(═O)₂(Q₁₁), —P(═O)(Q₁)(Q₁₂), or any combination thereof.

In an embodiment, R₁ may be:
deuterium, —F, —Cl, —Br, or —I, or
a methyl group, an ethyl group, a propyl group, or a butyl group, each unsubstituted or substituted with deuterium, —F, —Cl, —Br, —I, or any combination thereof.

In an embodiment, n1 may be an integer from 1 to 7.

In an embodiment, the organic compound may be represented by one of Formulae 1-1 to 1-3:

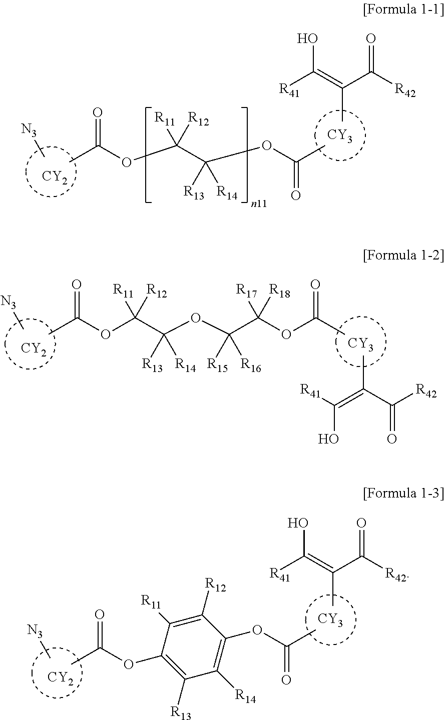

In Formulae 1-1 to 1-3,
R₁₁ to R₁₈ may each independently be: hydrogen; or as defined in connection with R₁ in Formula 1,
n11 may be an integer from 1 to 3,
when n11 is 2 or more, a plurality of R₁₁ to a plurality of R₁₄ may be respectively identical to or different from each other, and
ring CY₂, ring CY₃, R₄₁, and R₄₂ may each be as defined herein.

In an embodiment, the organic compound may be one of Compounds 1, 2, and 4 to 25:

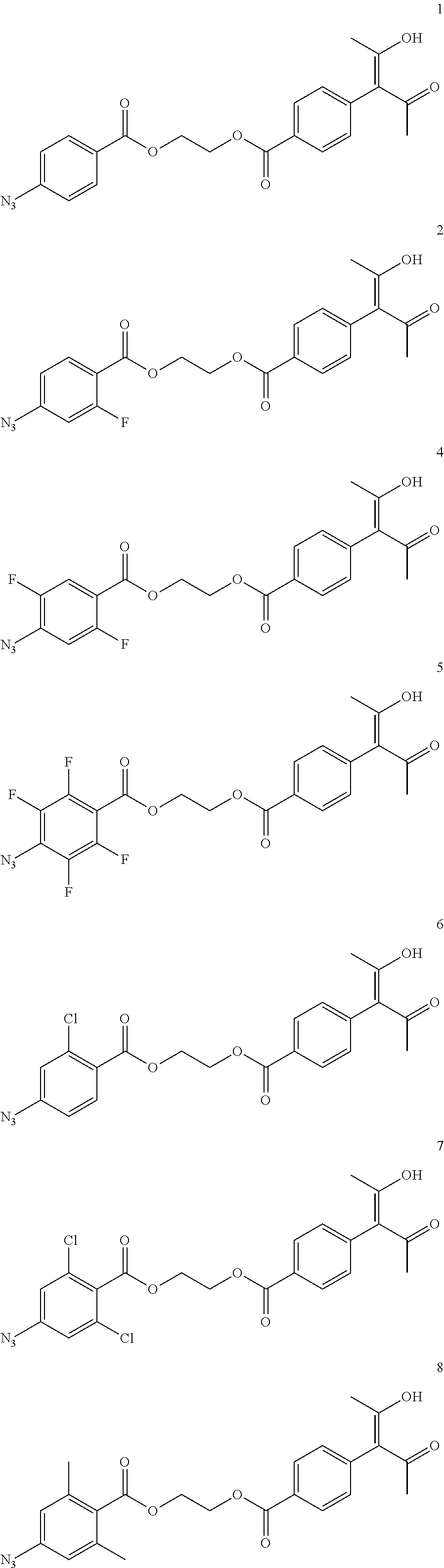

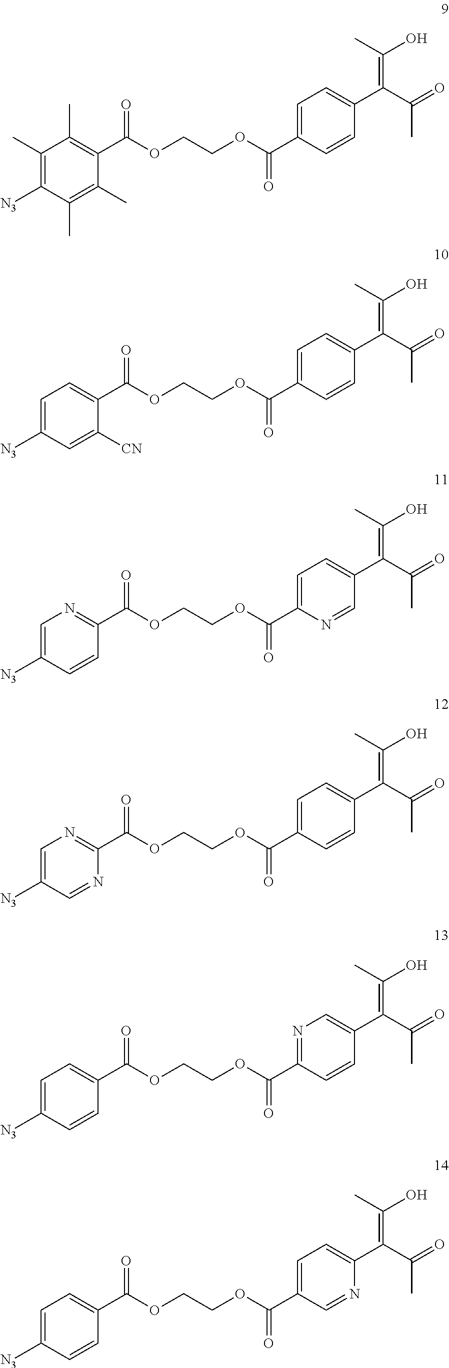 [SMILES: CC(=O)/C(=C(/C)O)c1ccc(C(=O)OCCOC(=O)c2c(C)c(C)c(N=[N+]=[N-])c(C)c2C)cc1 ; CC(=O)/C(=C(/C)O)c1ccc(C(=O)OCCOC(=O)c2ccc(N=[N+]=[N-])cc2C#N)cc1 ; CC(=O)/C(=C(/C)O)c1ccc(C(=O)OCCOC(=O)c2ccc(N=[N+]=[N-])cn2)nc1 ; CC(=O)/C(=C(/C)O)c1ccc(C(=O)OCCOC(=O)c2ncc(N=[N+]=[N-])cn2)cc1 ; CC(=O)/C(=C(/C)O)c1ccc(C(=O)OCCOC(=O)c2ccc(N=[N+]=[N-])cc2)nc1 ; CC(=O)/C(=C(/C)O)c1ccc(C(=O)OCCOC(=O)c2ccc(N=[N+]=[N-])cc2)cn1]

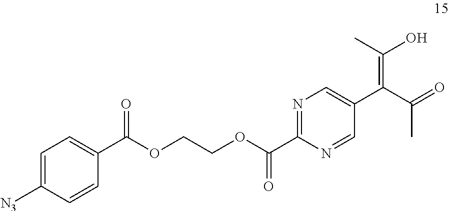 [SMILES: CC(=O)/C(=C(/C)O)c1cnc(C(=O)OCCOC(=O)c2ccc(N=[N+]=[N-])cc2)nc1]

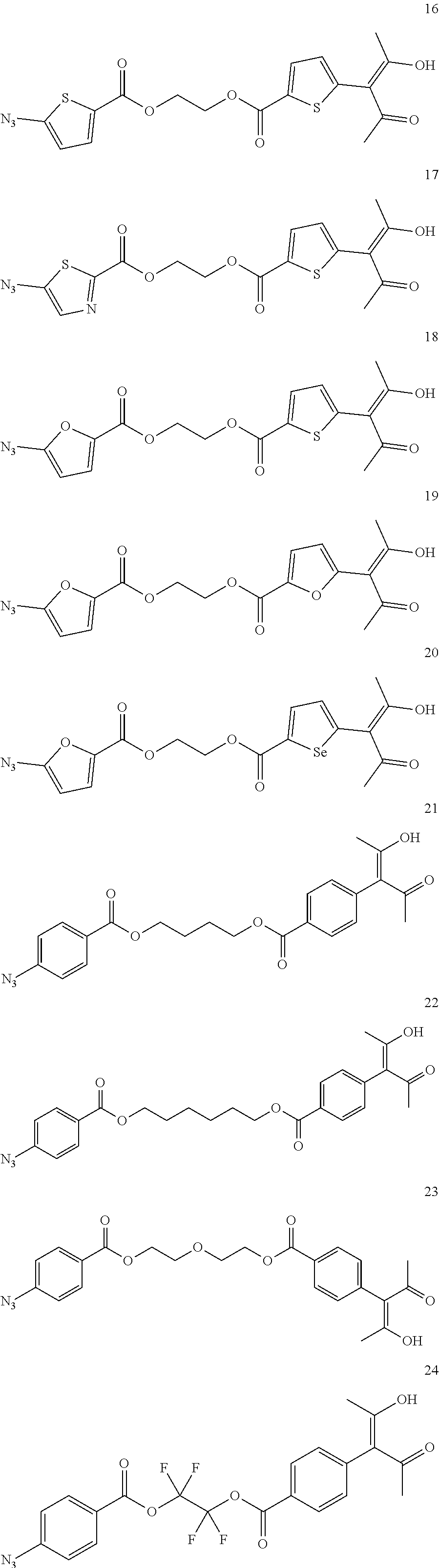

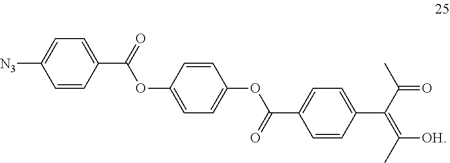 [SMILES: CC(=O)/C(=C(/C)O)c1ccc(C(=O)Oc2ccc(OC(=O)c3ccc(N=[N+]=[N-])cc3)cc2)cc1]

In an embodiment, the interlayer of the optoelectronic device may include a hole transport region, a photoactive layer, an electron transport region, or any combination thereof. The electron transport region may include a buffer layer, a hole blocking layer, an electron control layer, an electron transport layer, an electron injection layer, or any combination thereof. The organic compound or the organic ligand may be included in the electron transport region. For example, the organic compound or the organic ligand may be included in the electron transport layer.

In an embodiment, the optoelectronic device may include two or more organic compounds each independently represented by Formula 1, and an azide group of one of the two or more organic compounds may form at least one cross-link with an azide group of another of the two or more organic compounds.

In an embodiment, the optoelectronic device may further include metal oxide particles, wherein the metal oxide particles may form at least one coordinate bond with the organic compound or the organic ligand.

In an embodiment, the interlayer of the optoelectronic device may include a hole transport region, a photoactive layer, an electron transport region, or any combination thereof. The electron transport region may include a buffer layer, a hole blocking layer, an electron control layer, an electron transport layer, an electron injection layer, or any combination thereof. The metal oxide particles may be included in the electron transport region. For example, the organic compound or the organic ligand, and the metal oxide particles may be included in the electron transport region. For example, the metal oxide particles may be included in the electron transport layer.

In an embodiment, the metal oxide particles may be ZnO, ZrO₂, TiO₂, Nb₂O₅, Al₂O₃, SnO₂, or AZO.

Another embodiment provides an electronic apparatus which may include the optoelectronic device. The electronic apparatus may further include a thin-film transistor. For example, the electronic apparatus may further include a thin-film transistor including a source electrode and a drain electrode, and the first electrode of the optoelectronic device may be electrically connected to the source electrode or the drain electrode. In an embodiment, the electronic apparatus may further include a color filter, a color conversion layer, a touch screen layer, a polarizing layer, or any combination thereof. More details of the electronic apparatus may be referred to the descriptions provided herein.

In an embodiment, the electronic apparatus may further include a thin-film transistor electrically connected to the first electrode of the optoelectronic device, an emission layer between the first electrode and the second electrode of the optoelectronic device, and a color filter, a color conversion layer, a touch screen layer, a polarizing layer, or any combination thereof.

Another embodiment provides an electronic equipment which may include the electronic apparatus. The electronic equipment may be a flat panel display, a curved display, a computer monitor, a medical monitor, a television, an advertisement board, an indoor light, an outdoor light, a signaling light, a head-up display, a fully transparent display, a partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a microdisplay, a three-dimensional (3D) display, a virtual reality display, an augmented reality display, a vehicle, a video wall including multiple displays tiled together, a theater screen, a stadium screen, a phototherapy device, or a sign.

Another embodiment provides the organic compound which may be represented by Formula 1 and the organic ligand which may be represented by Formula 1A or Formula 1B.

Another embodiment provides an organic semiconductor thin film which may include: an organic compound represented by Formula 1 or an organic ligand represented by Formula 1A or Formula 1B; and metal oxide particles. For example, the metal oxide particles may be ZnO, ZrO₂, TiO₂, Nb₂O₅, Al₂O₃, SnO₂, or AZO.

In embodiments, in the organic semiconductor thin film, the metal oxide particles may form at least one coordinate bond with the organic compound or the organic ligand. For example, metal atoms of the metal oxide particles may form at least one coordinate bond with an acetyl acetone group of an organic ligand.

In an embodiment, the organic semiconductor thin film may include two or more organic compounds each independently represented by Formula 1, and an azide group of one of the two or more organic compounds may form at least one cross-link with an azide group of another of the two or more organic compounds.

Another embodiment provides a method of manufacturing an organic semiconductor thin film, and the method may include: preparing a solution including the organic compound described above; preparing a mixture by applying the solution to metal oxide particles; and irradiating the mixture with light. For example, the light may be ultraviolet (UV) light.

Due to the inclusion of the organic compound represented by Formula 1 or the organic ligand represented by Formula 1A or Formula 1B, the organic semiconductor thin film may have increased crystallinity and reduced defects. This may be because, in the case of the organic ligand which forms a coordinate bond with a metal oxide, an azide group of the organic ligand may form a cross-linking bond with an azide group of another organic compound or an azide group of another organic ligand.

Thus, in the organic semiconductor thin film, leakage current may be suppressed, and recombination of electrons and holes may be suppressed. As a result, an optoelectronic device including the organic semiconductor thin film may exhibit high photocurrent and low dark current.

Accordingly, a high-quality electronic apparatus and electronic equipment may be manufactured.

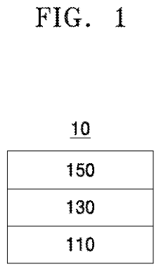

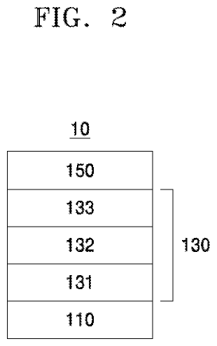

### [Description of FIGS. 1 and 2]

FIGS. 1 and 2 are each a schematic cross-sectional view of an optoelectronic device 10 according to an embodiment. However, a structure of an electronic device is not limited to the structures of FIGS. 1 and 2, and may be a structure of the related art other than the structures of FIGS. 1 and 2, depending on the type of electronic device.

Referring to FIG. 1, an optoelectronic device 10 according to an embodiment may include: a first electrode 110; a second electrode 150 facing the first electrode 110; and an interlayer 130 between the first electrode 110 and the second electrode 150.

In an embodiment, an interlayer 130 may include the organic compound or the organic ligand. In an embodiment, at least one of the first electrode 110 and the second electrode 150 may include the organic compound or the organic ligand.

For example, referring to FIG. 2, the interlayer 130 of the optoelectronic device 10 according to an embodiment may further include: a hole transport region 131 arranged on the first electrode 110; a photoactive layer 132 arranged on the hole transport region 131; and an electron transport region 133 arranged on the photoactive layer 132. The electron transport region 133 may be arranged between the photoactive layer 132 and the second electrode 150.

In the optoelectronic device 10, in case that light is incident from the side of the first electrode 110 and/or the second electrode 150 and the photoactive layer 132 absorbs light in a given wavelength range, excitons may be generated inside the photoactive layer 132. The excitons may be separated into holes and electrons in the photoactive layer 132, and the separated holes may move to the second electrode 150 or the first electrode 110. Here, the separated electrons may move to the first electrode 110 or the second electrode 150.

The optoelectronic device 10 may be, as a device that receives light, a photovoltaic cell, a photodetector, an organic solar cell, a photodiode, or a phototransistor.

Hereinafter, the structure of the optoelectronic device 10 according to an embodiment and a method of manufacturing the optoelectronic device 10 will be described in connection with FIGS. 1 and 2.

### [First Electrode 110]

In FIG. 1, a substrate may be further included under the first electrode 110 or on the second electrode 150. In an embodiment, as the substrate, a glass substrate or a plastic substrate may be used. The substrate may be a flexible substrate. For example, the substrate may include plastic having excellent heat resistance and durability, such as polyimide, polyethylene terephthalate (PET), polycarbonate, polyethylene naphthalate, polyarylate (PAR), polyetherimide, or any combination thereof.

The first electrode 110 may be formed by depositing or sputtering a material for forming the first electrode 110 on the substrate. The first electrode 110 may be an anode.

The first electrode 110 may be a reflective electrode, a semi-transmissive electrode, or a transmissive electrode. In an embodiment, in case that the first electrode 110 is a transmissive electrode, a material for forming the first electrode 110 may include indium tin oxide (ITO), indium zinc oxide (IZO), tin oxide (SnO₂), zinc oxide (ZnO), or any combination thereof. In embodiments, in case that the first electrode 110 is a semi-transmissive electrode or a reflective electrode, a material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or any combination thereof.

The first electrode 110 may have a structure consisting of a single layer or a structure including multiple layers. For example, the first electrode 110 may have a three-layer structure of ITO/Ag/ITO.

### [Interlayer 130]

The interlayer 130 may be arranged on the first electrode 110. The interlayer 130 may include the photoactive layer 132.

The interlayer 130 may further include the hole transport region 131 arranged between the first electrode 110 and the photoactive layer 132 and the electron transport region 133 arranged between the photoactive layer 132 and the second electrode 150.

The interlayer 130 may include the organic compound or the organic ligand.

The interlayer 130 may have a multi-layer structure including a layer including the organic compound or the organic ligand. In embodiments, the interlayer 130 may have a single-layer structure including a layer including the organic compound or the organic ligand.

The interlayer 130 included in the optoelectronic device 10 may be formed in a selected region by using one or more suitable methods selected from thermal deposition, vacuum deposition, laser deposition, spin coating, spray coating, casting, drop casting, dipping, Langmuir-Blodgett (LB) deposition, ink-jet printing, screen printing, laser printing, imprinting, laser-induced thermal imaging, and the like.

### [Second Electrode 150]

The second electrode 150 may be arranged on the interlayer 130. The second electrode 150 may be a cathode. The second electrode 150 may include a material having a high work function, such as a metal, an alloy, an electrically conductive compound, or any combination thereof.

The second electrode 150 may include lithium (Li), silver (Ag), magnesium (Mg), aluminum (AI), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a semi-transmissive electrode, or a reflective electrode.

The second electrode 150 may have a single-layer structure or a multi-layer structure.

### [Electron Transport Region 133 in Interlayer 130]

The electron transport region 133 may be arranged on the photoactive layer 132. The electron transport region 133 may be further included between the photoactive layer 132 and the second electrode 150.

The electron transport region 133 may facilitate migration of electrons generated and separated in the photoactive layer 132, thereby improving efficiency.

The electron transport region 133 may include at least one selected from an electron injection layer that facilitates injection of electrons, an electron transport layer that facilitates transport of electrons, and a hole blocking layer that blocks migration of holes.

The electron transport region 133 may include, for example, an inorganic material, an organic material, or an organic-inorganic material. The inorganic material may be a metal oxide, such as Mo oxide, W oxide, or Ni oxide, and the organic material may be an organic compound having electronic characteristics.

The electron transport layer may include, for example, an electron extracting metal oxide.

In an embodiment, the electron transport layer may include Ti oxide, Zn oxide, In oxide, Sn oxide, W oxide, Nb oxide, Mo oxide, Mg oxide, Zr oxide, Sr oxide, Yr oxide, La oxide, V oxide, Al oxide, Y oxide, Sc oxide, Sm oxide, Ga oxide, SrTi oxide, or any combination thereof.

In embodiments, the electron transport layer may include ZnO, TiO₂, Nb₂O₅, Al₂O₃, ZrO₂, SnO₂, or AZO, but embodiments are not limited thereto.

The electron transport layer may include an organic monomer and/or an organic polymer, in addition to or instead of the electron extracting metal oxide described above.

In an embodiment, the electron transport layer may include the organic compound or the organic ligand. Details on the organic compound or the organic ligand may be referred to the descriptions provided herein.

### [Photoactive Layer 132 in Interlayer 130]

The photoactive layer 132 may be arranged on the hole transport region 131. The photoactive layer 132 may absorb light to generate excitons. The excitons may generate holes and electrons, and the generated holes and electrons may respectively move to a cathode and an anode. For example, the photoactive layer 132 may absorb light to generate an electrical signal. Thus, the optoelectronic device 10 including the photoactive layer 132 may serve as an optical sensor.

The photoactive layer 132 may include an electron donor and an electron acceptor.

The electron donor may include, for example, poly(3-hexylthiophene) (P3HT), poly([2,6′-4,8-di(5-ethylhexylthienyl)benzo[1,2-b;3,3-b]dithiophene){3-fluoro-2[(2-ethylhexyl)carbonyl]thieno[3,4-b]thiophenediyl} (PTB7-Th), poly[[4,8-bis[5-(2-ethylhexyl)-2-thienyl]benzo[1,2-b:4,5-b′]dithiophene-2,6-diyl]-2,5-thiophenediyl[5,7-bis(2-ethylhexyl)-4,8-dioxo-4H,8H-benzo[1,2-c:4,5-c′]dithiophene-1,3-diyl]] (PBDB-T), polysiloxane carbazole, polyaniline, polyethylene oxide, poly(1-methoxy-4-(O-disperse Red 1))-2,5-phenylene-vinylene, polyindole, polycarbazole, polypyridiazine, polyisothianaphthalene, polyphenylene sulfide, polyvinylpyridine, polythiophene, polyfluorene, polypyridine, or a derivative thereof.

The photoactive layer 132 may have a multi-layer structure including a layer including an electron acceptor and a layer including an electron donor. In embodiments, the photoactive layer 132 may have a single-layer structure or a multi-layer structure including one layer in which an electron acceptor and an electron donor are mixed.

### [Hole Transport Region 131 in Interlayer 130]

The photoactive layer 132 may be arranged on the first electrode 110, and the hole transport region 131 may be further included between the first electrode 110 and the photoactive layer 132.

The hole transport region 131 may facilitate migration of holes generated and separated in the photoactive layer 132, thereby improving efficiency.

The hole transport region 131 may include at least one selected from a hole injection layer that facilitates injection of holes, a hole transport layer that facilitates transport of holes, and an electron blocking layer that blocks migration of electrons.

The hole transport region 131 may include an organic semiconductor film, and may include, for example, poly(3,4-ethylenediocy-thiophene) doped with poly(styrenesulfonic acid) (PEDOT:PSS), N,N′-bis(3-methylphenyl)—N,N′-diphenyl-[1,1′-biphenyl]-4,4′-diamine (TPD), copper phthalocyanine (CuPc), vanadium oxide (V₂O₅), tungsten oxide (WOs), nickel oxide (NiO), molybdenum oxide (MoO₂, MoOs), copper oxide (CuO), ruthenium oxide (RuO₂), tungsten oxide doped with cerium (CeWOs), a polymer having a sulfonation group introduced at the terminus of a conjugated polyelectrolyte chain group, chlorobenzoic acid, and any combination thereof, but embodiments are not limited thereto.

In an embodiment, the hole transport region 131 may include the organic compound or the organic ligand. Details on the organic compound or the organic ligand may be referred to the descriptions provided herein.

### [Electronic Apparatus]

The optoelectronic device 10 may be included in various electronic apparatuses. For example, the electronic apparatus including the optoelectronic device may be a light-emitting apparatus, an authentication apparatus, and the like.

The electronic apparatus (for example, a light-emitting apparatus) may further include, in addition to the optoelectronic device and alight-emitting device, a color filter, a color conversion layer, or a color filter and a color conversion layer. The color filter and/or the color conversion layer may be arranged in at least one traveling direction of light emitted from the light-emitting device. For example, the light emitted from the light-emitting device may be blue light or white light. In an embodiment, the color conversion layer may include a quantum dot.

The electronic apparatus may include a first substrate. The first substrate may include subpixels, the color filter may include color filter areas respectively corresponding to the subpixels, and the color conversion layer may include color conversion areas respectively corresponding to the subpixels.

A pixel-defining film may be arranged between the subpixels to define each of the subpixels.

The color filter may further include color filter areas and light-shielding patterns arranged among the color filter areas, and the color conversion layer may further include color conversion areas and light-shielding patterns arranged among the color conversion areas.

The color filter areas (or the color conversion areas) may include a first area emitting first color light, a second area emitting second color light, and/or a third area emitting third color light, wherein the first color light, the second color light, and/or the third color light may have different maximum emission wavelengths from one another. For example, the first color light may be red light, the second color light may be green light, and the third color light may be blue light. In embodiments, the color filter areas (or the color conversion areas) may include quantum dots. The first area may include a red quantum dot, the second area may include a green quantum dot, and the third area may not include a quantum dot. The first area, the second area, and/or the third area may each further include a scatterer.

For example, the light-emitting device may emit first light, the first area may absorb the first light to emit a first-first color light, the second area may absorb the first light to emit a second-first color light, and the third area may absorb the first light to emit a third-first color light. In this regard, the first-first color light, the second-first color light, and the third-first color light may have different maximum emission wavelengths. In an embodiment, the first light may be blue light, the first-first color light may be red light, the second-first color light may be green light, and the third-first color light may be blue light.

The electronic apparatus may further include a thin-film transistor, in addition to the optoelectronic device as described above. The thin-film transistor may include a source electrode, a drain electrode, and an active layer, and one of the source electrode and the drain electrode may be electrically connected to one of the first electrode and the second electrode of the optoelectronic device.

The thin-film transistor may further include a gate electrode, a gate insulating film, or the like.

The active layer may include crystalline silicon, amorphous silicon, an organic semiconductor, an oxide semiconductor, or the like.

The electronic apparatus may further include a sealing portion that seals the optoelectronic device. The sealing portion may be arranged between the color filter and/or the color conversion layer and the optoelectronic device. The sealing portion may prevent ambient air and moisture from penetrating into the optoelectronic device.

The sealing portion may be a sealing substrate including a transparent glass substrate or a plastic substrate. The sealing portion may be a thin-film encapsulation layer including at least one layer of an organic layer and/or an inorganic layer. In case that the sealing portion is a thin-film encapsulation layer, the electronic apparatus may be flexible.

Various functional layers may be further included on the sealing portion, in addition to the color filter and/or the color conversion layer, according to the use of the electronic apparatus. Examples of the functional layers may include a touch screen layer, a polarizing layer, or the like. The touch screen layer may be a pressure-sensitive touch screen layer, a capacitive touch screen layer, or an infrared touch screen layer. The authentication apparatus may be, for example, a biometric authentication apparatus that authenticates an individual by using biometric information of a living body (for example, fingertips, pupils, etc.).

The authentication apparatus may further include, in addition to the optoelectronic device as described above, a biometric information collector.

The electronic apparatus may be applied to various displays, light sources, lighting, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, or endoscope displays), fish finders, various measuring instruments, meters (for example, meters for a vehicle, an aircraft, and a vessel), projectors, and the like.

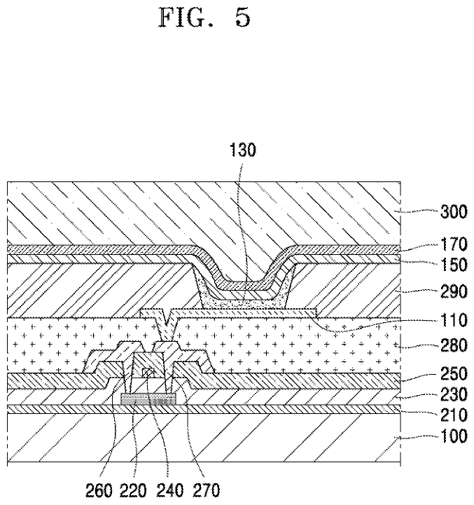

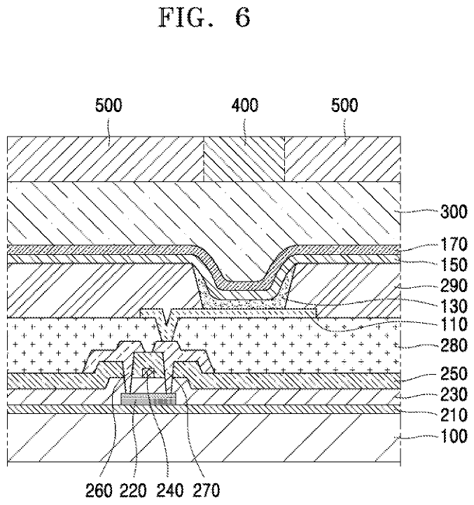

### [Description of FIGS. 5 and 6]

FIG. 5 is a schematic cross-sectional view of an electronic apparatus according to an embodiment.

The electronic apparatus of FIG. 5 may include a substrate 100, a thin-film transistor (TFT), an optoelectronic device, and an encapsulation portion 300 that seals the optoelectronic device.

The substrate 100 may be a flexible substrate, a glass substrate, or a metal substrate. A buffer layer 210 may be arranged on the substrate 100. The buffer layer 210 may prevent penetration of impurities through the substrate 100 and may provide a flat surface on the substrate 100.

A TFT may be arranged on the buffer layer 210. The TFT may include an active layer 220, a gate electrode 240, a source electrode 260, and a drain electrode 270.

The active layer 220 may include an inorganic semiconductor such as silicon or polysilicon, an organic semiconductor, or an oxide semiconductor, and may include a source region, a drain region, and a channel region.

A gate insulating film 230 for insulating the active layer 220 from the gate electrode 240 may be arranged on the active layer 220, and the gate electrode 240 may be arranged on the gate insulating film 230.

An interlayer insulating film 250 may be arranged on the gate electrode 240. The interlayer insulating film 250 may be arranged between the gate electrode 240 and the source electrode 260 and between the gate electrode 240 and the drain electrode 270, to insulate from one another.

The source electrode 260 and the drain electrode 270 may be arranged on the interlayer insulating film 250. The interlayer insulating film 250 and the gate insulating film 230 may be formed to expose the source region and the drain region of the active layer 220, and the source electrode 260 and the drain electrode 270 may respectively contact the exposed portions of the source region and the drain region of the active layer 220.

The TFT may be electrically connected to an optoelectronic device to drive the optoelectronic device, and may be covered and protected by a passivation layer 280. The passivation layer 280 may include an inorganic insulating film, an organic insulating film, or any combination thereof. An optoelectronic device may be provided on the passivation layer 280. The optoelectronic device may include the first electrode 110, the interlayer 130, and the second electrode 150.

The first electrode 110 may be arranged on the passivation layer 280. The passivation layer 280 may expose a portion of the drain electrode 270, and may not completely cover the drain electrode 270. The first electrode 110 may be electrically connected to the exposed portion of the drain electrode 270.

A pixel defining layer 290 including an insulating material may be arranged on the first electrode 110. The pixel defining layer 290 may expose a selected region of the first electrode 110, and the interlayer 130 may be formed in the exposed region of the first electrode 110. The pixel defining layer 290 may be a polyimide-based organic film or a polyacrylic-based organic film. Although not shown in FIG. 5, at least some layers of the interlayer 130 may extend beyond the upper portion of the pixel defining layer 290 to be provided in the form of a common layer.

The second electrode 150 may be arranged on the interlayer 130, and a capping layer 170 may be further included on the second electrode 150. The capping layer 170 may be formed to cover the second electrode 150.

The encapsulation portion 300 may be arranged on the capping layer 170. The encapsulation portion 300 may be arranged on an optoelectronic device to protect the optoelectronic device from moisture and/or oxygen. The encapsulation portion 300 may include: an inorganic film including silicon nitride (SiNx), silicon oxide (SiOx), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (for example, polymethyl methacrylate, polyacrylic acid, or the like), an epoxy-based resin (for example, aliphatic glycidyl ether (AGE), etc.), or any combination thereof; or a combination of the inorganic film and the organic film.

FIG. 6 is a schematic cross-sectional view of an electronic apparatus according to another embodiment.

The electronic apparatus of FIG. 6 may differ from the electronic apparatus of FIG. 5, at least in that a light-shielding pattern 500 and a functional region 400 are further included on the encapsulation portion 300. The functional region 400 may be a color filter area, a color conversion area, a light guide area, or any combination of the color filter area, the color conversion area, and the light guide area. In an embodiment, the optoelectronic device included in the electronic apparatus of FIG. 6 may be a tandem optoelectronic device.

### [Electronic Equipment]

The optoelectronic device may be included in various electronic equipment.

For example, the electronic equipment including the optoelectronic device may be a flat panel display, a curved display, a computer monitor, a medical monitor, a television, an advertisement board, an indoor light, an outdoor light, a signaling light, a head-up display, a fully transparent display, a partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a microdisplay, a three-dimensional (3D) display, a virtual reality display, an augmented reality display, a vehicle, a video wall including multiple displays tiled together, a theater screen, a stadium screen, a phototherapy device, or a sign.

Since the optoelectronic device has excellent photoelectric efficiency, the electronic equipment including the optoelectronic device may have characteristics such as high luminance, high resolution, and low power consumption.

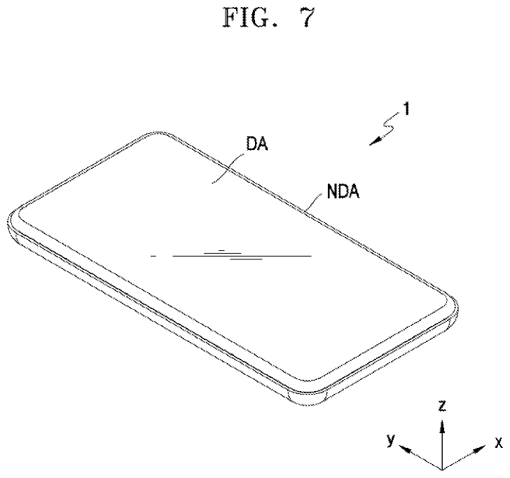

### [Description of FIG. 7]

FIG. 7 is a schematic perspective view of electronic equipment 1 including an optoelectronic device according to an embodiment.

The electronic equipment 1 may be, as an apparatus including an optical sensor, a portable electronic equipment, such as a mobile phone, a smartphone, a tablet personal computer (PC), a mobile communication terminal, an electronic notebook, an electronic book, a portable multimedia player (PMP), a navigation device, or an ultra-mobile PC (UMPC), but may also be various products, such as a television, a laptop, a monitor, a billboard, or an Internet of things (IOT). The electronic equipment 1 may be such a product as described above or a part thereof.

In an embodiment, the electronic equipment 1 may be a wearable device, such as a smart watch, a watch phone, a glasses-type display, or a head mounted display (HMD), or a part of the wearable device.

For convenience of explanation, FIG. 7 illustrates an embodiment in which the electronic equipment 1 is a smartphone.

The electronic equipment 1 may include a display area DA and a non-display area NDA outside the display area DA. A display apparatus may implement an image through a two-dimensional array of pixels that are arranged in the display area DA. An optoelectronic device may be arranged to absorb light incident through the display area DA or absorb light incident through the non-display area NDA.

The non-display area NDA may be an area that does not display an image, and may surround the display area DA. A driver for providing electrical signals or power to display devices arranged on the display area DA may be arranged in the non-display area NDA. A pad, which may be an area to which an electronic device or a printed circuit board may be electrically connected, may be arranged in the non-display area NDA.

In the electronic equipment 1, a length in an x-axis direction and a length in a y-axis direction may be different from each other. In an embodiment, as shown in FIG. 7, the length in the x-axis direction may be shorter than the length in the y-axis direction.

In another embodiment, the length in the x-axis direction may be the same as the length in the y-axis direction. In yet another embodiment, the length in the x-axis direction may be longer than the length in the y-axis direction.

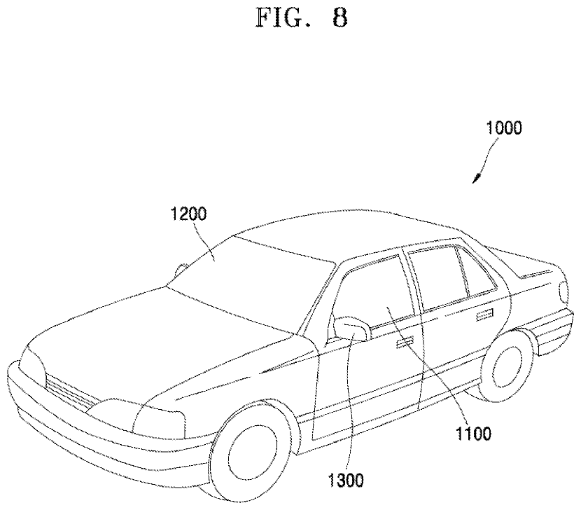

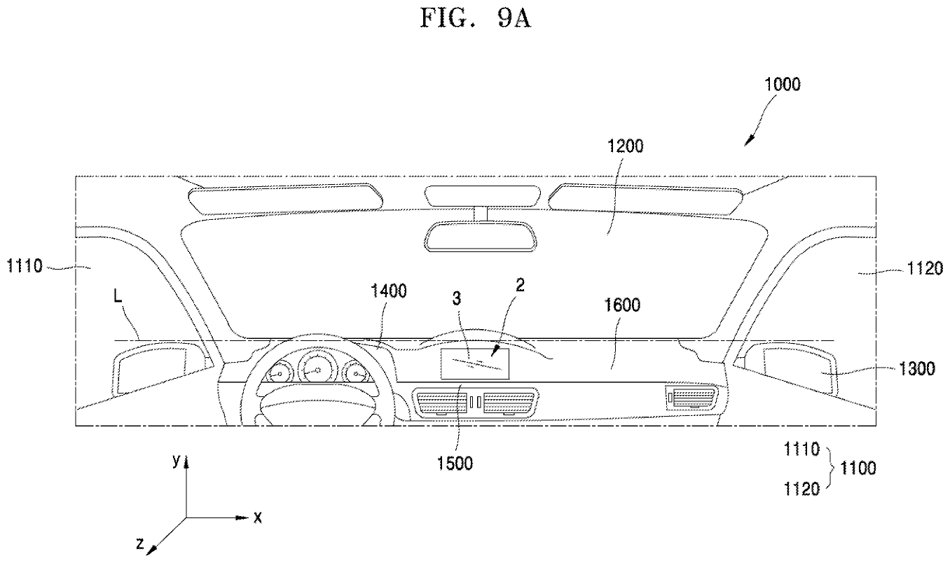

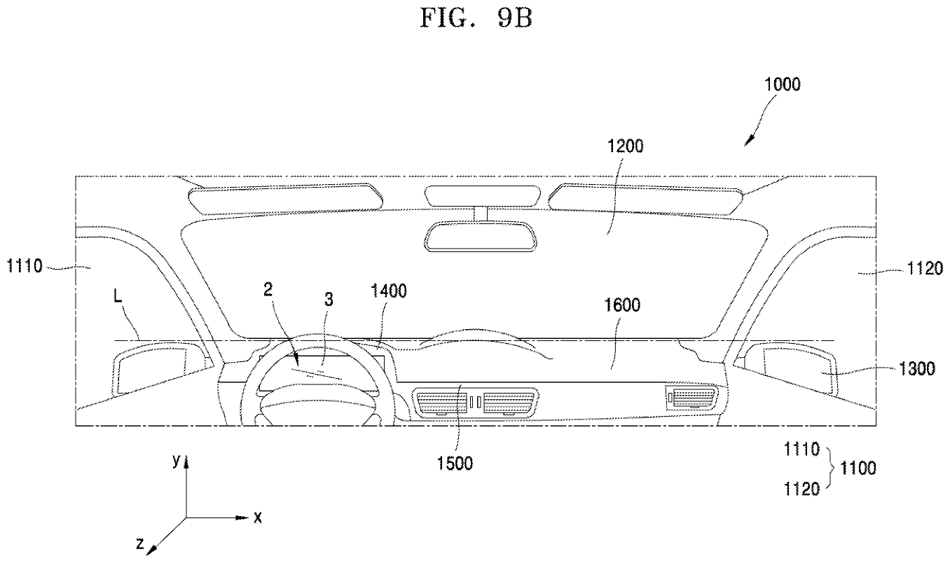

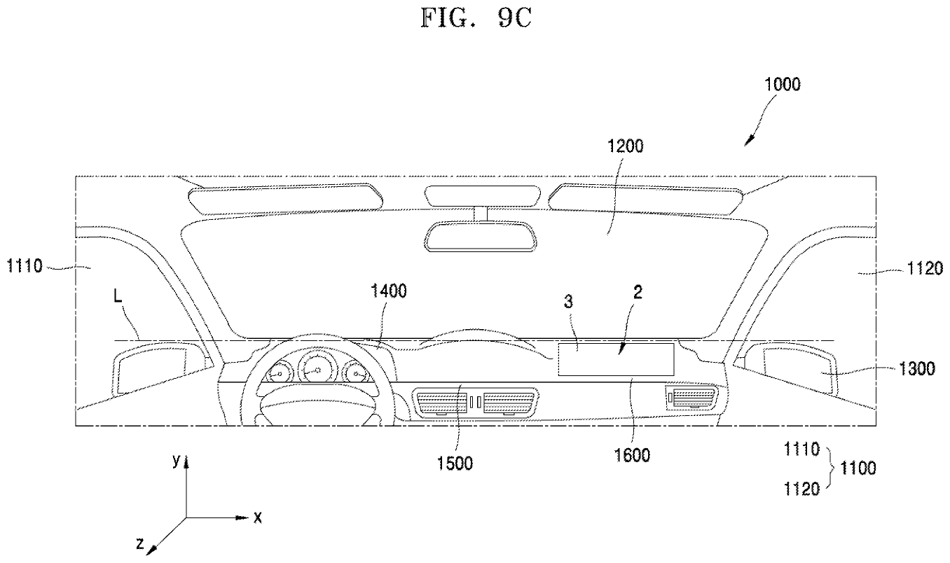

### [Description of FIGS. 8 and 9A to 9C]

FIG. 8 is a schematic view of the exterior of a vehicle 1000 as an electronic equipment including an optoelectronic device according to an embodiment. FIGS. 9A to 9C are each a schematic view of the interior of the vehicle 1000 according to an embodiment.

Referring to FIGS. 8, 9A, 9B, and 9C, the vehicle 1000 may refer to various apparatuses for moving a subject to be transported, such as a person, an object, or an animal, from a departure point to a destination point. Examples of the vehicle 1000 may include a vehicle traveling on a road or track, a vessel moving over a sea or river, an airplane flying in the sky using the action of air, and the like.

The vehicle 1000 may travel on a road or a track. The vehicle 1000 may move in a given direction according to the rotation of at least one wheel. Examples of the vehicle 1000 may include a three-wheeled or four-wheeled vehicle, a construction machine, a two-wheeled vehicle, a prime mover, a bicycle, and a train running on a track.

The vehicle 1000 may include a body having an interior and an exterior, and a chassis that is a portion excluding the body in which mechanical apparatuses necessary for driving are installed. The exterior of the body may include a front panel, a bonnet, a roof panel, a rear panel, a trunk, a pillar provided at a boundary between doors, and the like. The chassis of the vehicle 1000 may include a power generating apparatus, a power transmitting apparatus, a driving apparatus, a steering apparatus, a braking apparatus, a suspension apparatus, a transmission apparatus, a fuel apparatus, front, rear, left and right wheels, and the like.

The vehicle 1000 may include a side window glass 1100, a front window glass 1200, a side mirror 1300, a cluster 1400, a center fascia 1500, a passenger seat dashboard 1600, and a display apparatus 2.

The side window glass 1100 and the front window glass 1200 may be partitioned by a pillar arranged between the side window glass 1100 and the front window glass 1200.

The side window glass 1100 may be installed on the side of the vehicle 1000. In an embodiment, the side window glass 1100 may be installed in a door of the vehicle 1000. Multiple side window glasses 1100 may be provided and may face each other.

In an embodiment, the side window glass 1100 may include a first side window glass 1110 and a second side window glass 1120. In an embodiment, the first side window glass 1110 may be arranged adjacent to the cluster 1400, and the second side window glass 1120 may be arranged adjacent to the passenger seat dashboard 1600.

In an embodiment, the side window glasses 1100 may be spaced apart from each other in an x-direction or in an −x-direction. For example, the first side window glass 1110 and the second side window glass 1120 may be spaced apart from each other in the x direction or in the −x direction. An imaginary straight line L connecting the side window glasses 1100 to each other may extend in the x-direction or in the −x-direction. For example, the imaginary straight line L connecting the first side window glass 1110 and the second side window glass 1120 to each other may extend in the x direction or in the −x direction.

The front window glass 1200 may be installed on the front of the vehicle 1000. The front window glass 1200 may be arranged between the side window glasses 1100 facing each other.

The side mirror 1300 may provide a rear view of the vehicle 1000. The side mirror 1300 may be installed on the exterior of the body. In an embodiment, multiple side mirrors 1300 may be provided. Any one of the side mirrors 1300 may be arranged outside the first side window glass 1110. Another one of the side mirrors 1300 may be arranged outside the second side window glass 1120.

The cluster 1400 may be arranged in front of a steering wheel. The cluster 1400 may include a tachometer, a speedometer, a coolant thermometer, a fuel gauge, a turn signal indicator, a high beam indicator, a warning light, a seat belt warning light, an odometer, a driving record system, an automatic shift selector indicator light, a door open warning light, an engine oil warning light, and/or a low fuel warning light.

The center fascia 1500 may include a control panel on which buttons for adjusting an audio apparatus, an air conditioning apparatus, and a seat heater are arranged. The center fascia 1500 may be arranged on a side of the cluster 1400.

The passenger seat dashboard 1600 may be spaced apart from the cluster 1400 with the center fascia 1500 arranged therebetween. In an embodiment, the cluster 1400 may be arranged to correspond to a driver seat (not shown), and the passenger seat dashboard 1600 may be arranged to correspond to a passenger seat (not shown). In an embodiment, the cluster 1400 may be adjacent to the first side window glass 1110, and the passenger seat dashboard 1600 may be adjacent to the second side window glass 1120.

In an embodiment, the display apparatus 2 may include a display panel 3, and the display panel 3 may display an image. The display apparatus 2 may be arranged inside the vehicle 1000. In an embodiment, the display apparatus 2 may be arranged between the side window glasses 1100 facing each other. The display apparatus 2 may be arranged in at least one of the cluster 1400, the center fascia 1500, and the passenger seat dashboard 1600.

The display apparatus 2 may be an organic light-emitting display apparatus, an inorganic light-emitting display apparatus, a quantum dot display apparatus, or the like.

Referring to FIG. 9A, the display apparatus 2 may be arranged in the center fascia 1500. In an embodiment, the display apparatus 2 may display navigation information. In an embodiment, the display apparatus 2 may display information regarding audio settings, video settings, or vehicle settings.

Referring to FIG. 9B, the display apparatus 2 may be arranged in the cluster 1400. In case that the display apparatus 2 is arranged in the cluster 1400, the cluster 1400 may display driving information and the like through the display apparatus 2. For example, the cluster 1400 may digitally implement driving information. The cluster 1400 may digitally display vehicle information and driving information as images. For example, a needle and a gauge of a tachometer and various warning lights or icons may be displayed by digital signals.

Referring to FIG. 9C, the display apparatus 2 may be arranged in the passenger seat dashboard 1600. The display apparatus 2 may be embedded in the passenger seat dashboard 1600 or arranged on the passenger seat dashboard 1600. In an embodiment, the display apparatus 2 arranged on the passenger seat dashboard 1600 may display an image related to information displayed on the cluster 1400 and/or information displayed on the center fascia 1500. In embodiments, the display apparatus 2 arranged on the passenger seat dashboard 1600 may display information that is different from information displayed on the cluster 1400 and/or different from information displayed on the center fascia 1500.

### [Manufacturing Method]

Respective layers included in the hole transport region, the photoactive layer, and respective layers included in the electron transport region may be formed in a selected region by using one or more suitable methods selected from vacuum deposition, spin coating, casting, LB deposition, ink-jet printing, laser printing, laser-induced thermal imaging, and the like.

In case that respective layers included in the hole transport region, the photoactive layer, and respective layers included in the electron transport region are formed by vacuum deposition, the deposition may be performed at a deposition temperature of about 100° C. to about 500° C., a vacuum degree of about 10⁻⁸ torr to about 10⁻³ torr, and a deposition rate of about 0.01 Å/sec to about 100 Å/sec, depending on a material to be included in a layer to be formed and the structure of a layer to be formed.

### [Definitions of Terms]

The term “C₃-C₆₀ carbocyclic group” as used herein may be a cyclic group consisting of carbon atoms as the only ring-forming atoms and having 3 to 60 carbon atoms, and the term “C₁-C₆₀ heterocyclic group” as used herein may be a cyclic group that has 1 to 60 carbon atoms and further has, in addition to a carbon atom, at least one heteroatom as a ring-forming atom. The C₃-C₆₀ carbocyclic group and the C₁-C₆₀ heterocyclic group may each independently be a monocyclic group consisting of one ring or a polycyclic group in which two or more rings are condensed with each other. For example, a C₁-C₆₀ heterocyclic group may have 3 to 61 ring-forming atoms.

The term “cyclic group” as used herein may be a C₃-C₆₀ carbocyclic group or a C₁-C₆₀ heterocyclic group.

The term “π electron-rich C₃-C₆₀ cyclic group” as used herein may be a cyclic group that has 3 to 60 carbon atoms and may not include *—N═*′ as a ring-forming moiety. The term “r electron-deficient nitrogen-containing C₁-C₆₀ cyclic group” as used herein may be a heterocyclic group that has 1 to 60 carbon atoms and may include *—N═*′ as a ring-forming moiety.

In Embodiments,
a C₃-C₆₀ carbocyclic group may be a T1 group or a cyclic group in which at least two T1 groups are condensed with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),
the C₁-C₆₀ heterocyclic group may be a T2 group, a group in which at least two T2 groups are condensed with each other, or a group in which at least one T2 group and at least one T1 group are condensed with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),
a π electron-rich C₃-C₆₀ cyclic group may be a T1 group, a group in which at least two T1 groups are condensed with each other, a T3 group, a group in which at least two T3 groups are condensed with each other, or a group in which at least one T3 group and at least one T1 group are condensed with each other (for example, the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, etc.),
a π electron-deficient nitrogen-containing C₁-C₆₀ cyclic group may be a T4 group, a group in which at least two T4 groups are condensed with each other, a group in which at least one T4 group and at least one T1 group are condensed with each other, a group in which at least one T4 group and at least one T3 group are condensed with each other, or a group in which at least one T4 group, at least one T1 group, and at least one T3 group are condensed with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.), wherein
the T1 group may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbomane (or bicyclo[2.2.1]heptane) group, a norbornene group, a bicyclo[1.1.1]pentane group, a bicyclo[2.1.1]hexane group, a bicyclo[2.2.2]octane group, or a benzene group,
the T2 group may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrmidine group, a tetrahydropyrimidine group, a dihydropyrimidine group, a piperazine group, a tetrahydropyrazine group, a dihydropyrazine group, a tetrahydropyridazine group, or a dihydropyridazine group,
the T3 group may be a furan group, a thiophene group, a 1H-pyrrole group, a silole group, or a borole group, and
the T4 group may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “cyclic group”, “C₃-C₆₀ carbocyclic group”, “C₁-C₆₀ heterocyclic group”, “π electron-rich C₃-C₆₀ cyclic group”, or “π electron-deficient nitrogen-containing C₁-C₆₀ cyclic group” as used herein may each be a group condensed to any cyclic group, a monovalent group, or a polyvalent group (for example, a divalent group, a trivalent group, a tetravalent group, etc.) according to the structure of a formula for which the corresponding term is used. For example, a “benzene group” may be a benzo group, a phenyl group, a phenylene group, or the like, which may be readily understood by one of ordinary skill in the art according to the structure of a formula including the “benzene group.”

Examples of a monovalent C₃-C₆₀ carbocyclic group or a monovalent C₁-C₆₀ heterocyclic group may include a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group. Examples of a divalent C₃-C₆₀ carbocyclic group or a divalent C₁-C₆₀ heterocyclic group may include a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a divalent non-aromatic condensed heteropolycyclic group.

The term “C₁-C₆₀ alkyl group” as used herein may be a linear or branched aliphatic hydrocarbon monovalent group that has 1 to 60 carbon atoms, and examples thereof may include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, a tert-decyl group, and the like. The term “C₁-C₆₀ alkylene group” as used herein may be a divalent group having a same structure as the C₁-C₆₀ alkyl group.

The term “C₂-C₆₀ alkenyl group” as used herein may be a monovalent hydrocarbon group having at least one carbon-carbon double bond in the middle or at a terminus of a C₂-C₆₀ alkyl group, and examples thereof may include an ethenyl group, a propenyl group, a butenyl group, and the like. The term “C₂-C₆₀ alkenylene group” as used herein may be a divalent group having a same structure as the C₂-C₆₀ alkenyl group.

The term “C₂-C₆₀ alkynyl group” as used herein may be a monovalent hydrocarbon group having at least one carbon-carbon triple bond in the middle or at a terminus of a C₂-C₆₀ alkyl group, and examples thereof may include an ethynyl group, a propynyl group, and the like. The term “C₂-C₆₀ alkynylene group” as used herein may be a divalent group having a same structure as the C₂-C₆₀ alkynyl group.

The term “C₁-C₆₀ alkoxy group” as used herein may be a monovalent group represented by —O(A₁₀₁) (wherein A₁₀₁ may be a C₁-C₆₀ alkyl group), and examples thereof may include a methoxy group, an ethoxy group, an isopropyloxy group, and the like.

The term “C₃-C₁₀ cycloalkyl group” as used herein may be a monovalent saturated hydrocarbon cyclic group having 3 to 10 carbon atoms, and examples thereof may include a cyclopropyl group, a cyclobutyl group, a cyclopentyl group, a cyclohexyl group, a cycloheptyl group, a cyclooctyl group, an adamantanyl group, a norbomanyl group (or bicyclo[2.2.1]heptyl group), a bicyclo[1.1.1]pentyl group, a bicyclo[2.1.1]hexyl group, a bicyclo[2.2.2]octyl group, and the like. The term “C₃-C₁₀ cycloalkylene group” as used herein may be a divalent group having a same structure as the C₃-C₁₀ cycloalkyl group.

The term “C₁-C₁₀ heterocycloalkyl group” as used herein may be a monovalent cyclic group of 1 to 10 carbon atoms, further including, in addition to carbon atoms, at least one heteroatom, as ring-forming atoms, and examples thereof may include a 1,2,3,4-oxatriazolidinyl group, a tetrahydrofuranyl group, a tetrahydrothiophenyl group, and the like. The term “C₁-C₁₀ heterocycloalkylene group” as used herein may be a divalent group having a same structure as the C₁-C₁₀ heterocycloalkyl group.

The term “C₃-C₁₀ cycloalkenyl group” as used herein may be a monovalent cyclic group that has 3 to 10 carbon atoms and at least one carbon-carbon double bond in the ring thereof and no aromaticity, and examples thereof may include a cyclopentenyl group, a cyclohexenyl group, a cycloheptenyl group, and the like. The term “C₃-C₁₀ cycloalkenylene group” as used herein may be a divalent group having a same structure as the C₃-C₁₀ cycloalkenyl group.

The term “C₁-C₁₀ heterocycloalkenyl group” as used herein may be a monovalent cyclic group of 1 to 10 carbon atoms, further including, in addition to carbon atoms, at least one heteroatom, as ring-forming atoms, and having at least one carbon-carbon double bond in the cyclic structure thereof. Examples of a C₁-C₁₀ heterocycloalkenyl group may include a 4,5-dihydro-1,2,3,4-oxatriazolyl group, a 2,3-dihydrofuranyl group, a 2,3-dihydrothiophenyl group, and the like. The term “C₁-C₁₀ heterocycloalkenylene group” as used herein may be a divalent group having a same structure as the C₁-C₁₀ heterocycloalkenyl group.

The term “C₆-C₆₀ aryl group” as used herein may be a monovalent group having a carbocyclic aromatic system of 6 to 60 carbon atoms, and the term “C₆-C₆₀ arylene group” as used herein may be a divalent group having a carbocyclic aromatic system of 6 to 60 carbon atoms. Examples of a C₆-C₆₀ aryl group may include a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, an ovalenyl group, and the like. In case that the C₆-C₆₀ aryl group and the C₆-C₆₀ arylene group each include two or more rings, the respective rings may be condensed with each other.

The term “C₁-C₆₀ heteroaryl group” as used herein may be a monovalent group having a heterocyclic aromatic system of 1 to 60 carbon atoms, further including, in addition to carbon atoms, at least one heteroatom, as ring-forming atoms. The term “C₁-C₆₀ heteroarylene group” as used herein may be a divalent group having a heterocyclic aromatic system of 1 to 60 carbon atoms, further including, in addition to carbon atoms, at least one heteroatom, as ring-forming atoms. Examples of a C₁-C₆₀ heteroaryl group may include a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, a naphthyridinyl group, and the like. In case that the C₁-C₆₀ heteroaryl group and the C₁-C₆₀ heteroarylene group each include two or more rings, the respective rings may be condensed with each other.

The term “monovalent non-aromatic condensed polycyclic group” as used herein may be a monovalent group (for example, having 8 to 60 carbon atoms) having two or more rings condensed to each other, only carbon atoms as ring-forming atoms, and no aromaticity in its entire molecular structure. Examples of a monovalent non-aromatic condensed polycyclic group may include an indenyl group, a fluorenyl group, a spiro-bifluorenyl group, a benzofluorenyl group, an indenophenanthrenyl group, an indeno anthracenyl group, and the like. The term “divalent non-aromatic condensed polycyclic group” as used herein may be a divalent group having a same structure as the monovalent non-aromatic condensed polycyclic group.

The term “monovalent non-aromatic condensed heteropolycyclic group” as used herein may be a monovalent group (for example, having 1 to 60 carbon atoms) having two or more rings condensed to each other, further including, in addition to carbon atoms, at least one heteroatom, as ring-forming atoms, and having non-aromaticity in its entire molecular structure. Examples of a monovalent non-aromatic condensed heteropolycyclic group may include a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indeno carbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, a benzothienodibenzothiophenyl group, and the like. The term “divalent non-aromatic condensed heteropolycyclic group” as used herein may be a divalent group having a same structure as the monovalent non-aromatic condensed heteropolycyclic group.

The term “C₆-C₆₀ aryloxy group” as used herein may be a group represented by —O(A₁₀₂) (wherein A₁₀₂ may be a C₆-C₆₀ aryl group), and the term “C₆-C₆₀ arylthio group” as used herein may be a group represented by —S(A₁₀₃) (wherein A₁₀₃ may be a C₆-C₆₀ aryl group).

The term “C₇-C₆₀ arylalkyl group” as used herein may be a group represented by -(A₁₀₄)(A₁₀₅) (wherein A₁₀₄ may be a C₁-C₅₄ alkylene group, and A₁₀₅ may be a C₆-C₅₉ aryl group), and the term “C₂-C₆₀ heteroarylalkyl group” as used herein may be a group represented by -(A₁₀₆)(A₁₀₇) (wherein A₁₀₆ may be a C₁-C₅₉ alkylene group, and A₁₀₇ may be a C₁-C₅₉ heteroaryl group).

The term “heteroatom” as used herein may be any atom other than a carbon atom or a hydrogen atom. Examples of a heteroatom may include O, S, N, P, Si, B, Ge, Se, and any combination thereof.

The term “Ph” as used herein refers to a phenyl group, the term “Me” as used herein refers to a methyl group, the term “Et” as used herein refers to an ethyl group, the terms “tert-Bu” or “Bu^{t}” as used herein each refers to a tert-butyl group, and the term “OMe” as used herein refers to a methoxy group.

The term “biphenyl group” as used herein may be a “phenyl group substituted with a phenyl group.” For example, the “biphenyl group” may be a substituted phenyl group having a C₆-C₆₀ aryl group as a substituent.

The term “terphenyl group” as used herein may be a “phenyl group substituted with a biphenyl group.” For example, the “terphenyl group” may be a substituted phenyl group having, as a substituent, a C₆-C₆₀ aryl group substituted with a C₆-C₆₀ aryl group.

The symbols * and *′ as used herein, unless defined otherwise, each refer to a binding site to a neighboring atom in a corresponding formula or moiety.

The terms “x-axis”, “y-axis”, and “z-axis” as used herein are not limited to three axes in an orthogonal coordinate system (e.g., a Cartesian coordinate system), and may be interpreted in a broader sense than the aforementioned three axes in an orthogonal coordinate system. For example, the x-axis, y-axis, and z-axis may describe axes that are orthogonal to each other, or may describe axes that are in different directions that are not orthogonal to each other.

Hereinafter, compounds according to embodiments and optoelectronic devices according to embodiments will be described in detail with reference to Synthesis Examples and Examples. The wording “B was used instead of A” used in describing Synthesis Examples may be that an identical molar equivalent of B was used in place of A.

### EXAMPLES

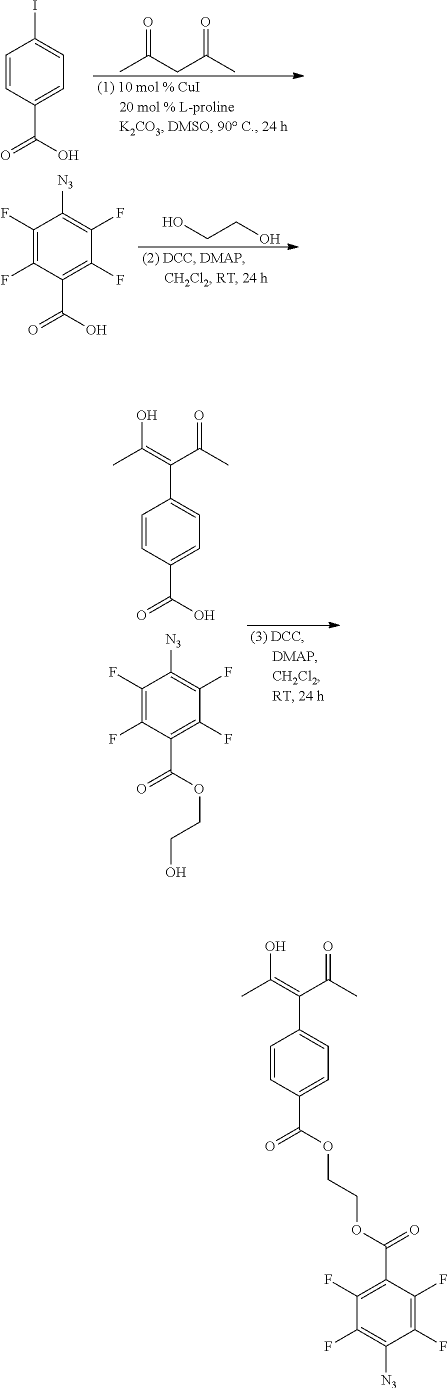

### Synthesis Example 1: Synthesis of Organic Compound 5 (2-((4-(2-hydroxy-4-oxopent-2-en-3-yl)benzoyl)oxy)ethyl 4-azido-2,3,5,6-tetrafluorobenzoate)

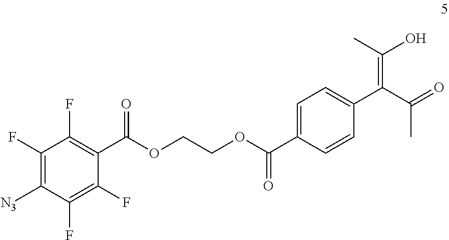 [SMILES: CC(=O)/C(=C(/C)O)c1ccc(C(=O)OCCOC(=O)c2c(F)c(F)c(N=[N+]=[N-])c(F)c2F)cc1]

### (1) Synthesis of 2-hydroxyethyl 4-azido-2,3,5,6-tetrafluorobenzoate

A mixture of 4-azido-2,3,5,6-tetrafluorobenzoic acid (1 g, 4.25 mmol), ethylene glycol (2.3 mL, 42.53 mmol), and anhydrous dichloromethane (25 mL) was stirred at 298 K, and DMAP (51 mg, 0.42 mmol) was added thereto. After 30 minutes, the temperature was lowered to 0° C., and DCC (1 M in dichloromethane) (4.67 mL, 4.67 mmol) was added thereto under continuous N₂ purging. After 24 hours, the reaction mixture was neutralized using water. An extraction process was performed thereon using dichloromethane, followed by drying using MgSO₄. A solvent was removed therefrom by evaporation under reduced pressure. The resulting crude product was purified by silica gel column chromatography using ethyl acetate/n-hexane (2/5) as an eluent. 2-hydroxyethyl 4-azido-2,3,5,6-tetrafluorobenzoate was obtained as a colorless liquid (yield of 65%).

### (2) Synthesis of 4-(2-hydroxy-4-oxopent-2-en-3-yl)benzoic acid

A mixture of 4-iodobenzoic acid (1.1 g, 4.5 mmol), acetylacetone (1.4 mL, 13.5 mmol), K₂CO₃ (3.1 g, 22.5 mmol), Cul (0.25 g, 1.35 mmol), and L-proline (0.1 g, 0.9 mmol) dissolved in 15 mL of DMSO was stirred at 298 K for 5 minutes and heated at 90° C. for 24 hours under continuous nitrogen purging. After 24 hours, the reaction solution was cooled and poured into a 1M HCl solution. After the pH was adjusted to neutral conditions, an extraction process was performed thereon using ethyl acetate. An organic layer extracted therefrom was dried using MgSO₄, and a solvent was removed therefrom under vacuum. The resulting crude product was purified by silica gel column chromatography using a mixture of n-hexane/ethyl acetate (1:1). 3-(4-benzoic acid)pentane-2,4-dione was obtained (yield of 54%).

### (3) Synthesis of 2-((4-(2-hydroxy-4-oxopent-2-en-3-yl)benzoyl)oxy)ethyl 4-azido-2,3,5,6-tetrafluorobenzoate (Compound 5)

A mixture of 2-hydroxyethyl 4-azido-2,3,5,6-tetrafluorobenzoate (0.35 g, 1.25 mmol) and 3-(4-benzoic acid)pentane-2,4-dione (0.25 g, 1.14 mmol) dissolved in anhydrous dichloromethane(25 mL) was stirred at 298 K, and DMAP (13 mg, 0.11 mmol) was added thereto. After 30 minutes, the temperature was lowered to 0° C., and DCC (1 M in dichloromethane) (1.25 mL, 1.25 mmol) was added thereto under continuous N₂ purging. After 24 hours, the reaction mixture was neutralized using water. An extraction process was performed thereon using dichloromethane, followed by drying using MgSO₄. A solvent was removed therefrom by evaporation under reduced pressure. The resulting crude product was purified by silica gel column chromatography using ethyl acetate/n-hexane (1/5) as an eluent. Compound 5 was obtained as a pale yellow solid (340 mg, yield of 56%).

### Synthesis Example 2: Synthesis of Organic Compound 1 (2-((4-azidobenzoyl)oxy)ethyl-4-(2-hydroxy-4-oxopent-2-en-3-yl)benzoate)

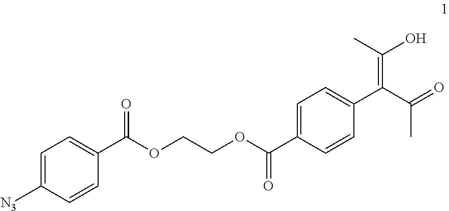 [SMILES: CC(=O)/C(=C(/C)O)c1ccc(C(=O)OCCOC(=O)c2ccc(N=[N+]=[N-])cc2)cc1]

Compound 1 was synthesized in the same manner as used to synthesize Compound 5, except that 2-hydroxyethyl 4-azidobenzoate was used instead of 2-hydroxyethyl 4-azido-2,3,5,6-tetrafluorobenzoate.

### Synthesis Example 3: Synthesis of Organic Compound 8 (2-((4-(2-hydroxy-4-oxopent-2-en-3-v)benzoyl)oxy)ethyl 4-azido-2,6-dimethylbenzoate)

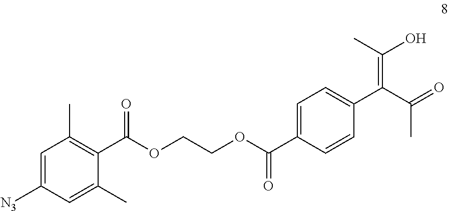 [SMILES: CC(=O)/C(=C(/C)O)c1ccc(C(=O)OCCOC(=O)c2c(C)cc(N=[N+]=[N-])cc2C)cc1]

Compound 8 was synthesized in the same manner as used to synthesize Compound 5, except that 2-hydroxyethyl 4-azido-2,6-dimethylbenzoate was used instead of 2-hydroxyethyl 4-azido-2,3,5,6-tetrafluorobenzoate.

### Synthesis Example 4: Synthesis of Organic Compound 10 (2-((4-(2-hydroxy-4-oxopent-2-en-3-yl)benzoyl)oxy)ethyl 4-azido-2-cyanobenzoate)

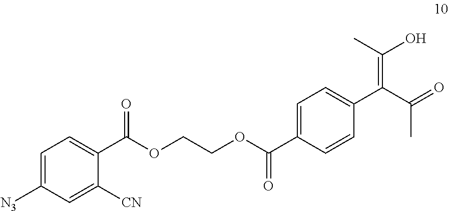 [SMILES: CC(=O)/C(=C(/C)O)c1ccc(C(=O)OCCOC(=O)c2ccc(N=[N+]=[N-])cc2C#N)cc1]

Compound 10 was synthesized in the same manner as used to synthesize Compound 5, except that 2-hydroxyethyl 4-azido-2-cyanobenzoate was used instead of 2-hydroxyethyl 4-azido-2,3,5,6-tetrafluorobenzoate.

### Synthesis Example 5: Synthesis of Organic Compound 11 (2-((5-azidopicolinoyl)oxy)ethyl-5-(2-hydroxy-4-oxopent-2-en-3-yl)picolinate)

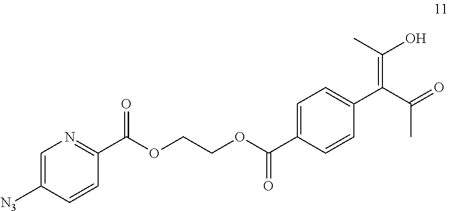 [SMILES: CC(=O)/C(=C(/C)O)c1ccc(C(=O)OCCOC(=O)c2ccc(N=[N+]=[N-])cn2)cc1]

Compound 11 was synthesized in the same manner as used to synthesize Compound 5, except that 2-hydroxyethyl 5-azidopicolinate was used instead of 2-hydroxyethyl 4-azido-2,3,5,6-tetrafluorobenzoate, and 5-(2-hydroxy-4-oxopent-2-en-3-yl)picolinic acid was used instead of 4-(2-hydroxy-4-oxopent-2-en-3-yl)benzoic acid.

### Synthesis Example 6: Synthesis of Organic Compound 12 (2-((4-(2-hydroxy-4-oxopent-2-en-3-yl)benzoyl)oxy)ethyl-5-azidopyrimidine-2-carboxylate)

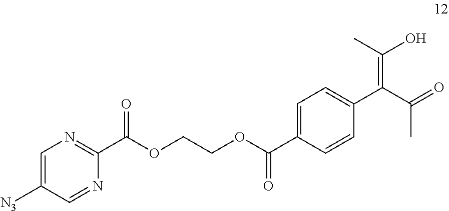 [SMILES: CC(=O)/C(=C(/C)O)c1ccc(C(=O)OCCOC(=O)c2ncc(N=[N+]=[N-])cn2)cc1]

Compound 12 was synthesized in the same manner as used to synthesize Compound 5, except that 2-hydroxyethyl 5-azidopyrimidine-2-carboxylate was used instead of 2-hydroxyethyl 4-azido-2,3,5,6-tetrafluorobenzoate.

### Synthesis Example 7: Synthesis of Organic Compound 15 (2-((4-azidobenzoyl)oxy)ethyl-5-(2-hydroxy-4-oxopent-2-en-3-yl)pyrimidine-2-carboxylate)

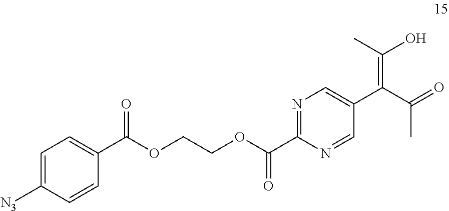 [SMILES: CC(=O)/C(=C(/C)O)c1cnc(C(=O)OCCOC(=O)c2ccc(N=[N+]=[N-])cc2)nc1]

Compound 15 was synthesized in the same manner as used to synthesize Compound 5, except that 5-(2-hydroxy-4-oxopent-2-en-3-yl)pyrimidine-2-carboxylic acid was used instead of 4-(2-hydroxy-4-oxopent-2-en-3-yl)benzoic acid.

### Synthesis Example 8: Synthesis of Organic Compound 19 (2-((5-azidofuran-2-carbonyl)oxy)ethyl-5-(2-hydroxy-4-oxopent-2-en-3-yl)furan-2-carboxylate)

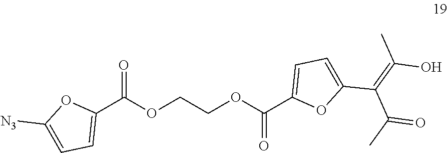 [SMILES: CC(=O)/C(=C(/C)O)c1ccc(C(=O)OCCOC(=O)c2ccc(N=[N+]=[N-])o2)o1]

Compound 19 was synthesized in the same manner as used to synthesize Compound 5, except that, 2-hydroxyethyl 5-azidofuran-2-carboxylate was used instead of 2-hydroxyethyl 4-azido-2,3,5,6-tetrafluorobenzoate, and 5-(2-hydroxy-4-oxopent-2-en-3-yl)furan-2-carboxylic acid was used instead of 4-(2-hydroxy-4-oxopent-2-en-3-yl)benzoic acid.

### Synthesis Example 9: Synthesis of Organic Compound 22 (6-((4-azidobenzoyl)oxy)hexyl-4-(2-hydroxy-4-oxopent-2-en-3-yl)benzoate)

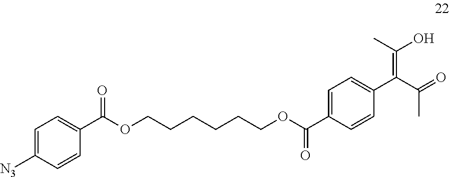 [SMILES: CC(=O)/C(=C(/C)O)c1ccc(C(=O)OCCCCCCOC(=O)c2ccc(N=[N+]=[N-])cc2)cc1]

Compound 22 was synthesized in the same manner as used to synthesize Compound 5, except that 6-hydroxyhexyl 4-azidobenzoate was used instead of 2-hydroxyethyl 4-azido-2,3,5,6-tetrafluorobenzoate.

### Synthesis Example 10: Synthesis of Organic Compound 23 (2-(2-((4-azidobenzoyl)oxy)ethoxy)ethyl-4-(2-hydroxy-4-oxopent-2-en-3-yl)benzoate)

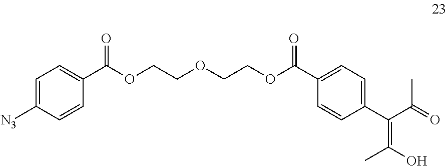 [SMILES: CC(=O)/C(=C(/C)O)c1ccc(C(=O)OCCOCCOC(=O)c2ccc(N=[N+]=[N-])cc2)cc1]

Compound 23 was synthesized in the same manner as used to synthesize Compound 5, except that 2-(2-hydroxyethoxy)ethyl 4-azidobenzoate was used instead of 2-hydroxyethyl 4-azido-2,3,5,6-tetrafluorobenzoate.

### Synthesis Example 11: Synthesis of Organic Compound 24 (2-((4-azidobenzoyl)oxy)-1,1,2,2-tetrafluoroethyl-4-(2-hydroxy-4-oxopent-2-en-3-yl)benzoate)

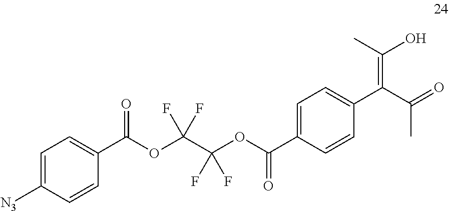 [SMILES: CC(=O)/C(=C(/C)O)c1ccc(C(=O)OC(F)(F)C(F)(F)OC(=O)c2ccc(N=[N+]=[N-])cc2)cc1]

Compound 24 was synthesized in the same manner as used to synthesize Compound 5, except that 1,1,2,2-tetrafluoro-2-hydroxyethyl 4-azidobenzoate was used instead of 2-hydroxyethyl 4-azido-2,3,5,6-tetrafluorobenzoate.

### Synthesis Example 12: Synthesis of Organic Compound 25 (4-((4-azidobenzoyl)oxy)phenyl-4-(2-hydroxy-4-oxopent-2-en-3-yl)benzoate)

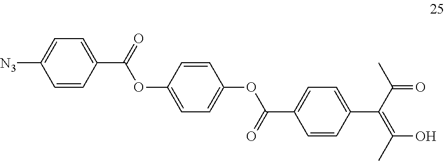 [SMILES: CC(=O)/C(=C(/C)O)c1ccc(C(=O)Oc2ccc(OC(=O)c3ccc(N=[N+]=[N-])cc3)cc2)cc1]

Compound 25 was synthesized in the same manner as used to synthesize Compound 5, except that 4-hydroxyphenyl 4-azidobenzoate was used instead of 2-hydroxyethyl 4-azido-2,3,5,6-tetrafluorobenzoate.

¹H NMR and MS/FAB of the compounds synthesized according to Synthesis Examples are shown in Table 1.

TABLE 1

| | | MS/FAB | |
|---|---|---|---|
| No. | ¹H NMR (CDCl₃, 500 MHZ) | found | calc. |
| Compound 1 | δ = 8.02(s, 2H), 7.67(d, 2H), 7.45(d, 2H), 7.35(s, | 409.13 | 409.40 |
| | 2H), 4.66(m, 4H), 2.29(s, 3H), 2.27(s, 3H) | | |
| Compound 5 | δ = 7.67(d, 2H), 7.45(d, 2H), 4.66(m, 4H), 2.29(s, | 481.09 | 481.36 |
| | 3H), 2.27(s, 3H) | | |
| Compound 8 | δ = 7.67(d, 2H), 7.45(d, 2H), 7.18(d, 2H), 4.66(m, | 437.16 | 437.45 |
| | 4H), 2.48(s, 6H), 2.29(s, 3H), 2.27(s, 3H) | | |
| Compound 10 | δ = 8.19(d, 1H), 7.69(s, 1H), 7.67(d, 2H), 7.63(d, | 434.12 | 434.41 |
| | 1H), 7.45(d, 2H), 4.66(m, 4H), 2.29(s, 3H), 2.27(s, | | |
| | 3H) | | |
| Compound 11 | δ = 8.77(s, 1H), 8.66(s, 1H), 8.29(d, 1H), 8.08(d, | 411.12 | 411.37 |
| | 2H), 6.13(d, 1H), 4.66(m, 4H), 2.29(s, 3H), 2.27(s, | | |
| | 3H) | | |
| Compound 12 | δ = 9.06(s, 2H), 7.67(d, 2H), 7.45(d, 2H), 4.66(m, | 411.12 | 411.37 |
| | 4H), 2.29(s, 3H), 2.27(s, 3H) | | |
| Compound 15 | δ = 8.89(s, 2H), 8.02(d, 2H), 7.24(d, 2H), 4.66(m, | 411.12 | 411.37 |
| | 4H), 2.29(s, 3H), 2.27(s, 3H) | | |
| Compound 19 | δ = 7.39(d, 2H), 6.93(s, 1H), 6.77(s, 1H), 4.66(m, | 389.09 | 389.32 |
| | 4H), 2.29(s, 3H), 2.27(s, 3H) | | |
| Compound 22 | δ = 8.02(s, 2H), 7.67(d, 2H), 7.45(d, 2H), 7.35(s, | 465.19 | 465.51 |
| | 2H), 4.33(m, 4H), 2.29(s, 3H), 2.27(s, 3H), 1.78(m, | | |
| | 4H), 1.43(m, 4H) | | |
| Compound 23 | δ = 8.02(s, 2H), 7.67(d, 2H), 7.45(d, 2H), 7.35(s, | 453.15 | 453.45 |
| | 2H), 4.37(m, 4H), 3.81(m, 4H), 2.29(s, 3H), 2.27(s, | | |
| | 3H) | | |
| Compound 24 | δ = 8.02(s, 2H), 7.67(d, 2H), 7.45(d, 2H), 7.35(s, | 481.09 | 481.36 |
| | 2H), 2.29(s, 3H), 2.27(s, 3H) | | |
| Compound 25 | δ = 8.19(d, 2H), 7.84(d, 2H), 7.49(d, 2H), 7.39(d, | 457.13 | 457.44 |
| | 2H), 7.36(d, 4H), 2.29(s, 3H), 2.27(s, 3H) | | |

### Example 1

An ITO glass substrate was continuously cleaned every 20 minutes in an ultrasonic bath by using detergent, deionized water, acetone, and isopropanol, and dried overnight in an oven at 80° C. The substrate was treated in an UV ozone generator for 30 minutes.

After a solution of 10 mL of methoxy ethanol, 1 g of zinc acetate, 50 mg of Compound 1, and 0.28 mL of ethanol amine was stirred at 60° C. for 12 hours, the solution was cross-linked on the ITO glass substrate by using an UV lamp (254 nm, 15 W) and annealed at 200° C. for 30 minutes, thereby forming a charge transport layer to which a novel ligand was applied.

P3HT was dissolved in 1-chlorobenzene (concentration of 20 mg/mL), 0.5 vol % of 1-chloronaphthalene as a solvent additive was added thereto, and the mixture was stirred at 80° C. for 12 hours and filtered through a 0.45 μm nylon filter, thereby preparing a photoactive solution.

60 mg/mL of the photoactive solution was spin-coated on the charge transport layer at 1,000 rpm for 60 seconds, and the resulting thin-film was annealed at 130° C. for 10 minutes in a glove box, thereby forming a photoactive layer.

A MoOx layer having a thickness of 30 nm and an Ag layer having a thickness of 100 nm were thermally deposited on the photoactive layer at a pressure of less than 10⁻⁵ Pa, thereby completing the manufacture of an optoelectronic device of Example 1.

### Examples 2 to 14

In Examples 2 to 12, optoelectronic devices were manufactured in the same manner as in Example 1, except that compounds introduced as organic ligands shown in Table 2 were each used instead of Compound 1.

In Example 13, an optoelectronic device was manufactured in the same manner as in Example 1, except that zirconium acetate was used instead of zinc acetate, and a compound introduced as an organic ligand shown in Table 2 was used instead of Compound 1.

In Example 14, an optoelectronic device was manufactured in the same manner as in Example 1, except that titanium acetate was used instead of zinc acetate, and a compound introduced as an organic ligand shown in Table 2 was used instead of Compound 1.

### Comparative Examples 1 to 3

Optoelectronic devices were manufactured in the same manner as in Examples 1, 13, and 14, respectively, except that an organic ligand according to the disclosure was not used.

### Evaluation Example 1: Evaluation of EQE and Dark Current of Optoelectronic Device

To evaluate characteristics of the optoelectronic devices manufactured in Examples 1 to 14 and Comparative Examples 1 to 3, external quantum efficiency (EQE) and dark current density (@-3V, E-08Acm⁻²) were measured, and results thereof are shown in Table 2.

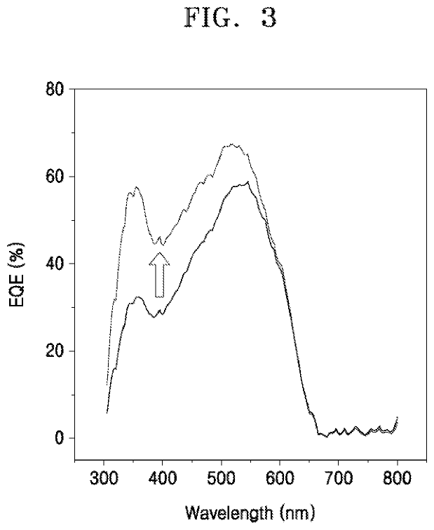

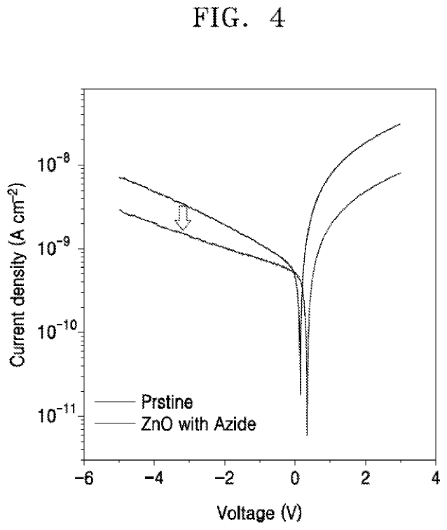

A graph showing external quantum efficiency (EQE) versus wavelength for the optoelectronic devices according to Example 2 and Comparative Example 1 is shown in FIG. 3. A graph showing dark current density versus voltage (J-V curve) of the optoelectronic devices according to Example 2 and Comparative Example 1 is shown in FIG. 4.

The EQE was measured by using IPCE measurement system equipment. The equipment was calibrated by using a Si photodiode. After the optoelectronic devices of Examples 1 to 14 were each installed in the equipment, the EQE in the wavelength range of about 300 nm to about 800 nm was measured.

The dark current (@-3V, E-08Acm⁻²) was measured using I-V measurement equipment.

TABLE 2

| | Charge transport layer | | | |
|---|---|---|---|---|
| | Metal oxide | Compound introduced | | Dark current |
| | particle | as organic ligand | EQE [%] | [@-3V, E-08Acm⁻²] |
| Example 1 | ZnO | Compound 1 | 61 | 2.34 |
| Example 2 | ZnO | Compound 5 | 65 | 2.74 |
| Example 3 | ZnO | Compound 8 | 64 | 2.61 |
| Example 4 | ZnO | Compound 10 | 62 | 2.65 |
| Example 5 | ZnO | Compound 11 | 59 | 2.14 |
| Example 6 | ZnO | Compound 12 | 61 | 2.32 |
| Example 7 | ZnO | Compound 15 | 57 | 2.41 |
| Example 8 | ZnO | Compound 19 | 58 | 2.29 |
| Example 9 | ZnO | Compound 22 | 64 | 2.57 |
| Example 10 | ZnO | Compound 23 | 62 | 2.68 |
| Example 11 | ZnO | Compound 24 | 63 | 2.34 |
| Example 12 | ZnO | Compound 25 | 61 | 2.96 |
| Example 13 | ZrO₂ | Compound 5 | 50 | 2.45 |
| Example 14 | TiO₂ | Compound 5 | 52 | 2.58 |
| Comparative | ZnO | None | 50 | 8.51 |
| Example 1 | | | | |
| Comparative | ZrO₂ | None | 38 | 8.42 |
| Example 2 | | | | |
| Comparative | TiO₂ | None | 41 | 7.98 |
| Example 3 | | | | |

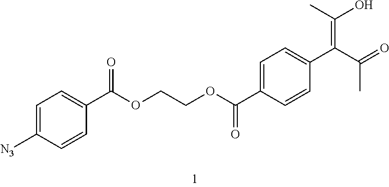 [SMILES: CC(=O)/C(=C(/C)O)c1ccc(C(=O)OCCOC(=O)c2ccc(N=[N+]=[N-])cc2)cc1]

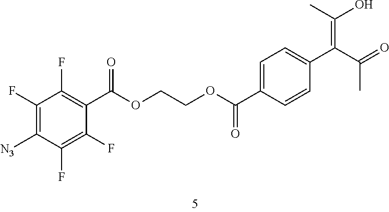 [SMILES: CC(=O)/C(=C(/C)O)c1ccc(C(=O)OCCOC(=O)c2c(F)c(F)c(N=[N+]=[N-])c(F)c2F)cc1]

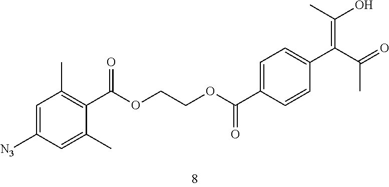 [SMILES: CC(=O)/C(=C(/C)O)c1ccc(C(=O)OCCOC(=O)c2c(C)cc(N=[N+]=[N-])cc2C)cc1]

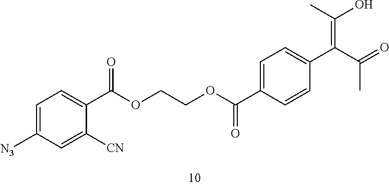 [SMILES: CC(=O)/C(=C(/C)O)c1ccc(C(=O)OCCOC(=O)c2ccc(N=[N+]=[N-])cc2C#N)cc1]

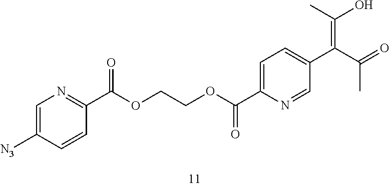 [SMILES: CC(=O)/C(=C(/C)O)c1ccc(C(=O)OCCOC(=O)c2ccc(N=[N+]=[N-])cn2)nc1]

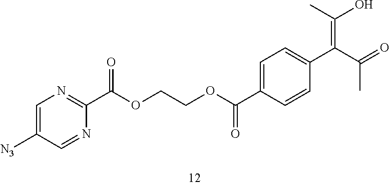 [SMILES: CC(=O)/C(=C(/C)O)c1ccc(C(=O)OCCOC(=O)c2ncc(N=[N+]=[N-])cn2)cc1]

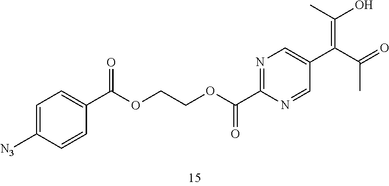 [SMILES: CC(=O)/C(=C(/C)O)c1cnc(C(=O)OCCOC(=O)c2ccc(N=[N+]=[N-])cc2)nc1]

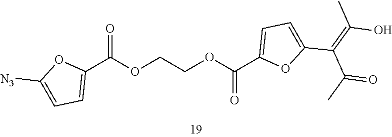 [SMILES: CC(=O)/C(=C(/C)O)c1ccc(C(=O)OCCOC(=O)c2ccc(N=[N+]=[N-])o2)o1]

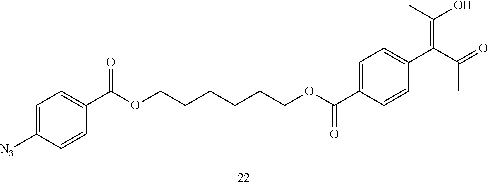 [SMILES: CC(=O)/C(=C(/C)O)c1ccc(C(=O)OCCCCCCOC(=O)c2ccc(N=[N+]=[N-])cc2)cc1]

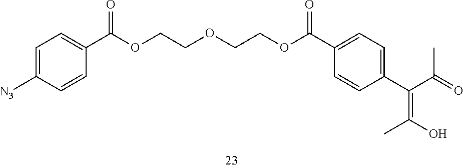 [SMILES: CC(=O)/C(=C(/C)O)c1ccc(C(=O)OCCOCCOC(=O)c2ccc(N=[N+]=[N-])cc2)cc1]

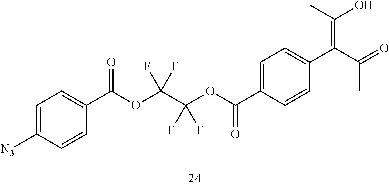 [SMILES: CC(=O)/C(=C(/C)O)c1ccc(C(=O)OC(F)(F)C(F)(F)OC(=O)c2ccc(N=[N+]=[N-])cc2)cc1]

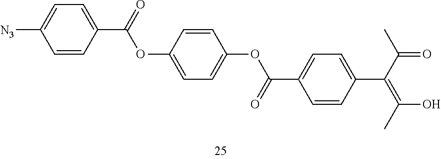 [SMILES: CC(=O)/C(=C(/C)O)c1ccc(C(=O)Oc2ccc(OC(=O)c3ccc(N=[N+]=[N-])cc3)cc2)cc1]

From Table 2, it was confirmed that the optoelectronic devices of Examples 1 to 14 had significantly high EQE values, but had significantly low dark current values, compared to the optoelectronic devices of Comparative Examples 1 to 3.

Referring to FIG. 3, it was confirmed that the optoelectronic device of Example 2 had significantly improved EQE compared to the optoelectronic device of Comparative Example 1.

Referring to FIG. 4, it was confirmed that the optoelectronic device of Example 2 had significantly reduced dark current compared to the optoelectronic device of Comparative Example 1.

According to embodiments, an organic semiconductor thin film including an organic compound represented by Formula 1 or an organic ligand represented by Formula 1A or 1B may have increased crystallinity and reduced defects. Accordingly, an optoelectronic device including the organic semiconductor thin film may have high photocurrent, and thus, a high-quality electronic apparatus and electronic equipment may be manufactured by using the optoelectronic device.

Embodiments have been disclosed herein, and although terms are employed, they are used and are to be interpreted in a generic and descriptive sense only and not for purposes of limitation. In some instances, as would be apparent to one of ordinary skill in the art, features, characteristics, and/or elements described in connection with an embodiment may be used singly or in combination with features, characteristics, and/or elements described in connection with other embodiments unless otherwise specifically indicated. Accordingly, it will be understood by those of ordinary skill in the art that various changes in form and details may be made without departing from the spirit and scope of the disclosure as set forth in the claims.

What is claimed is:

1. An optoelectronic device comprising:
a first electrode;
a second electrode facing the first electrode;
an interlayer between the first electrode and the second electrode; and
an organic compound represented by Formula 1 or an organic ligand represented by Formula 1A or Formula 1B: 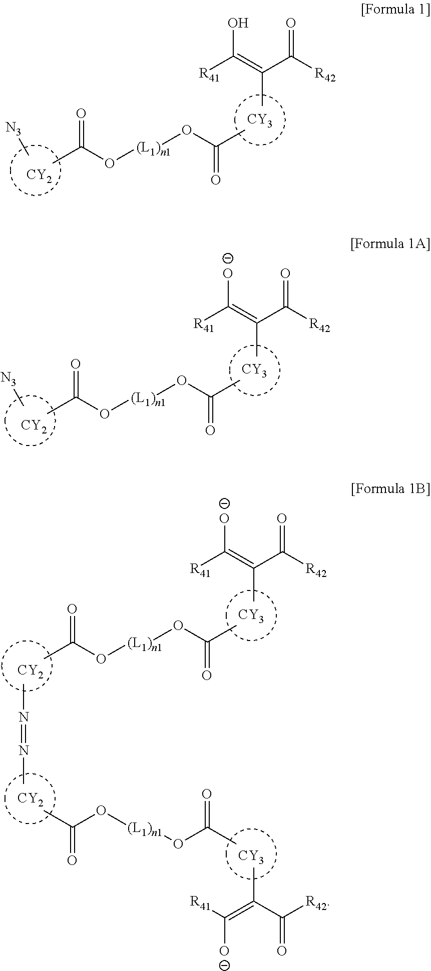
wherein in Formulae 1, 1A, and 1B,
ring CY₂ is a C₃-C₆₀ carbocyclic group or a C₁-C₆₀ heterocyclic group, each unsubstituted or substituted with at least one R₂,
ring CY₃ is a C₃-C₆₀ carbocyclic group or a C₁-C₆₀ heterocyclic group, each unsubstituted or substituted with at least one R₃,
L₁ is:
a single bond;
—O—*′; *—S—*′; or *—Se—*′; or
a C₁-C₆₀ alkylene group, a C₂-C₆₀ alkenylene group, a C₂-C₆₀ alkynylene group, a C₆-C₆₀ arylene group, or a C₁-C₆₀ heteroarylene group, each unsubstituted or substituted with at least one R₁,
* and *′ each indicate a binding site to a neighboring atom,
n1 is an integer from 0 to 10,
when n1 is 0, L₁ is a single bond,
when n1 is 2 or more, a plurality of L₁ are identical to or different from each other,
R₁, R₂, and R₃ are each independently:
deuterium, —F, —Cl, —Br, —I, a hydroxyl group, a cyano group, or a nitro group;
a C₁-C₆₀ alkyl group, a C₂-C₆₀ alkenyl group, a 2-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or a combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
R₄₁ and R₄₂ are each independently:
hydrogen, deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or a combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or a combination thereof.

2. The optoelectronic device of claim 1, wherein
the interlayer comprises a hole transport region, a photoactive layer, an electron transport region, or a combination thereof, and
the organic compound or the organic ligand is included in the electron transport region.

3. The optoelectronic device of claim 1, wherein
the optoelectronic device comprises two or more organic compounds each independently represented by Formula 1, and
an azide group of one of the two or more organic compounds forms at least one cross-link with an azide group of another of the two or more organic compounds.

4. The optoelectronic device of claim 1, further comprising metal oxide particles, wherein
the metal oxide particles form at least one coordinate bond with the organic compound or the organic ligand.

5. The optoelectronic device of claim 4, wherein
the interlayer comprises a hole transport region, a photoactive layer, an electron transport region, or a combination thereof, and
the organic compound or the organic ligand, and the metal oxide particles are included in the electron transport region.

6. The optoelectronic device of claim 4, wherein the metal oxide particles are ZnO, ZrO₂, TiO₂, Nb₂O₅, Al₂O₃, SnO₂, or AZO.

7. An electronic apparatus comprising the optoelectronic device of claim 1.

8. The electronic apparatus of claim 7, further comprising:
a thin-film transistor electrically connected to the first electrode of the optoelectronic device;
an emission layer between the first electrode and the second electrode of the optoelectronic device; and
a color filter, a color conversion layer, a touch screen layer, a polarizing layer, or a combination thereof.

9. An electronic equipment comprising the electronic apparatus of claim 7, wherein
the electronic equipment is a flat panel display, a curved display, a computer monitor, a medical monitor, a television, an advertisement board, an indoor light, an outdoor light, a signaling light, a head-up display, a fully transparent display, a partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a microdisplay, a three-dimensional (3D) display, a virtual reality display, an augmented reality display, a vehicle, a video wall including multiple displays tiled together, a theater screen, a stadium screen, a phototherapy device, or a sign.

10. An organic compound represented by Formula 1: 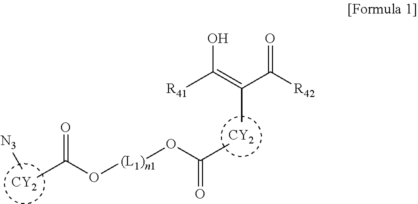
wherein in Formula 1,
ring CY₂ is a C₃-C₆₀ carbocyclic group or a C₁-C₆₀ heterocyclic group, each unsubstituted or substituted with at least one R₂,
ring CY₃ is a C₃-C₆₀ carbocyclic group or a C₁-C₆₀ heterocyclic group, each unsubstituted or substituted with at least one R₃,
L₁ is:
a single bond;
—O—*′; *—S—*′; or *—Se—*′; or
a C₁-C₆₀ alkylene group, a C₂-C₆₀ alkenylene group, a C₂-C₆₀ alkynylene group, a C₆-C₆₀ arylene group, or a C₁-C₆₀ heteroarylene group, each unsubstituted or substituted with at least one R₁,
* and *′ each indicate a binding site to a neighboring atom,
n1 is an integer from 0 to 10,
when n1 is 0, L₁ is a single bond,
when n1 is 2 or more, a plurality of L₁ are identical to or different from each other,
R₁, R₂, and R₃ are each independently:
deuterium, —F, —Cl, —Br, —I, a hydroxyl group, a cyano group, or a nitro group;
a C₁-C₆₀ alkyl group, a C₂-C₆₀ alkenyl group, a C₁-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or a combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
R₄₁ and R₄₂ are each independently:
hydrogen, deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or a combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁₁ to Q₁₃, Q₂₁ to Q₂₃, and Q₂₃ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or a combination thereof.

11. The organic compound of claim 10, wherein
ring CY₂ is a C₆-C₃ aryl group or a C₁-C₃₀ heteroaryl group, each substituted with at least one R₂, and
ring CY₃ is a C₆-C₃ aryl group or a C₁-C₃₀ heteroaryl group, each substituted with at least one R₃.

12. The organic compound of claim 10, wherein ring CY₂ is a moiety represented by one of Formulae 2-1 to 2-6: 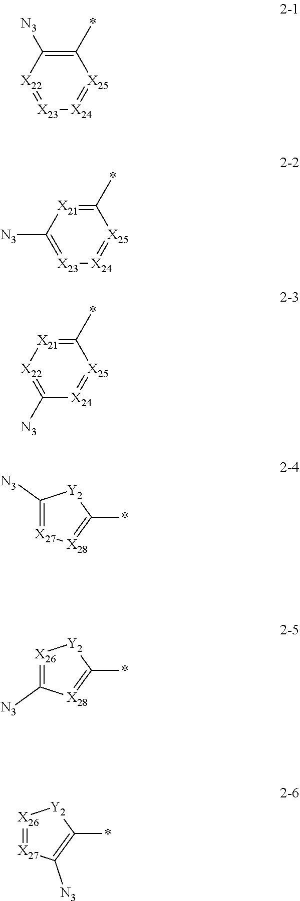
wherein in Formulae 2-1 to 2-6,
X₂₁ is N or C(R₂₁),
X₂₂ is N or C(R₂₂),
X₂₃ is N or C(R₂₃),
X₂₄ is N or C(R₂₄),
X₂₅ is N or C(R₂₅),
X₂₆ is N or C(R₂₆),
X₂₇ is N or C(R₂₇),
X₂₈ is N or C(R₂₈),
R₂₁ to R₂₈ are each independently:
hydrogen; or
as defined in connection with R₂ in Formula 1,
Y₂ is O, S, or Se, and
* indicates a binding site to a neighboring atom.

13. The organic compound of claim 10, wherein ring CY₃ is a moiety represented by one of Formulae 3-1 to 3-6: 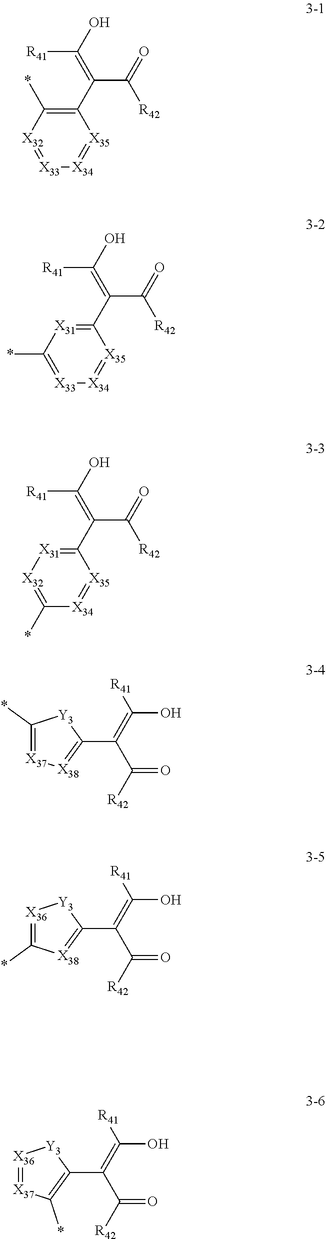
wherein in Formulae 3-1 to 3-6,
X₃₁ is N or C(R₃₁),
X₃₂ is N or C(R₃₂),
X₃₃ is N or C(R₃₃),
X₃₄ is N or C(R₃₄),
X₃₅ is N or C(R₃₅),
X₃₆ is N or C(R₃₆),
X₃₇ is N or C(R₃₇),
X₃₈ is N or C(R₃₈),
R₃₁ to R₃₈ are each independently:
hydrogen; or
as defined in connection with R₃ in Formula 1,
R₄₁ and R₄₂ are each as described in Formula 1,
Y₃ is O, S, or Se, and
* indicates a binding site to a neighboring atom.

14. The organic compound of claim 10, wherein
L₁ is *—O—*, *—S—*, *—C(R₁₁)(R₁₂)—*′, *—C(R₁₁)═(R₁₂)—*, *—C≡C*, or a phenylene group unsubstituted or substituted with at least one R₁,
R₁₁ and R₁₂ are each independently:
hydrogen; or
as defined in connection with R₁ in Formula 1,
R₁ is as described in Formula 1, and
* and *′ each indicate a binding site to a neighboring atom.

15. The organic compound of claim 10, wherein R₁, R₂, and R₃ are each independently:
deuterium, —F, —Cl, —Br, —I, a cyano group, or a nitro group; or
a C₁-C₂₀ alkyl group, a C₂-C₂₀ alkenyl group, a C₂-C₂₀ alkynyl group, or a C₂-C₂₀ alkoxy group, each unsubstituted or substituted with deuterium, —F, —Cl, —Br, —I, a cyano group, a nitro group, Si(Q₁₁)(Q₁₂)(Q₁₃), —B(Q₁₁)(Q₁₂), —C(═O)(Q₁₁), —S(═O)₂(Q₁₁), —P(═O)(Q₁₁)(Q₁₂), or a combination thereof.

16. The organic compound of claim 10, wherein the organic compound is represented by one of Formulae 1-1 to 1-3: 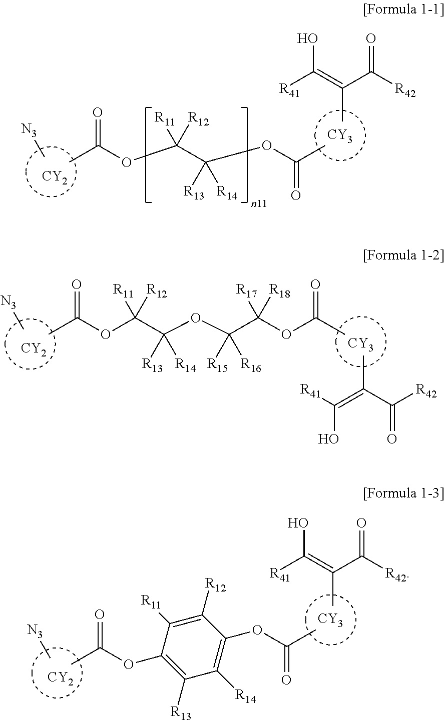
wherein in Formulae 1-1 to 1-3,
R₁₁ to R₁₈ are each independently:
hydrogen; or
as defined in connection with R₁ in Formula 1,
n11 is an integer from 1 to 3,
when n11 is 2 or more, a plurality of R₁₁ to a plurality of R₁₄ are respectively identical to or different from each other, and
ring CY₂, ring CY₃, R₄₁, and R₄₂ are each as defined in Formula 1.

17. The organic compound of claim 10, wherein the organic compound is one of Compounds 1, 2, and 4 to 25:

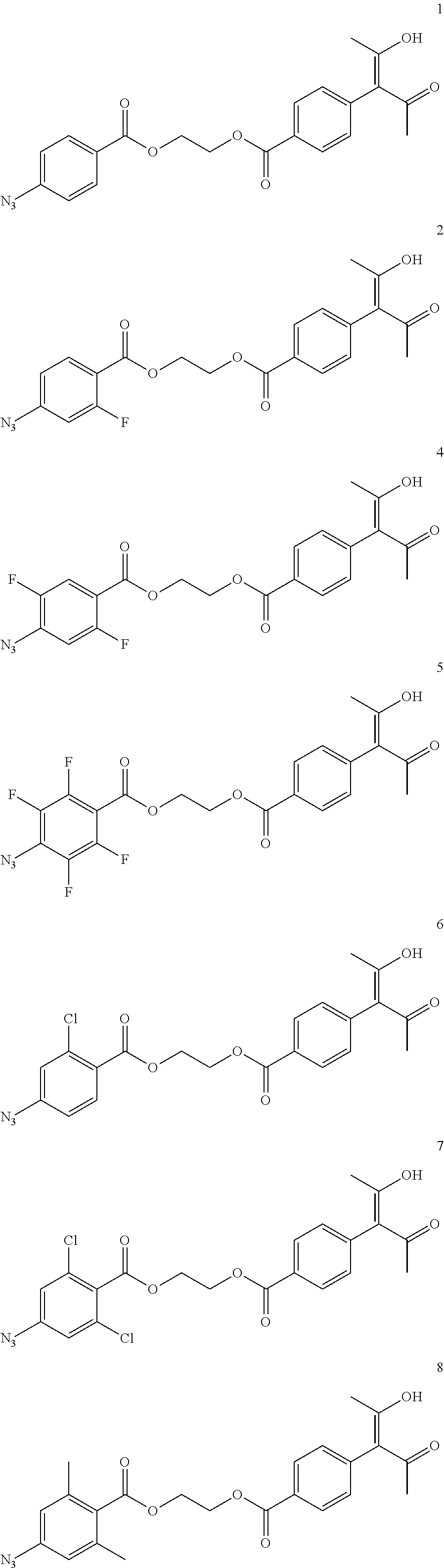

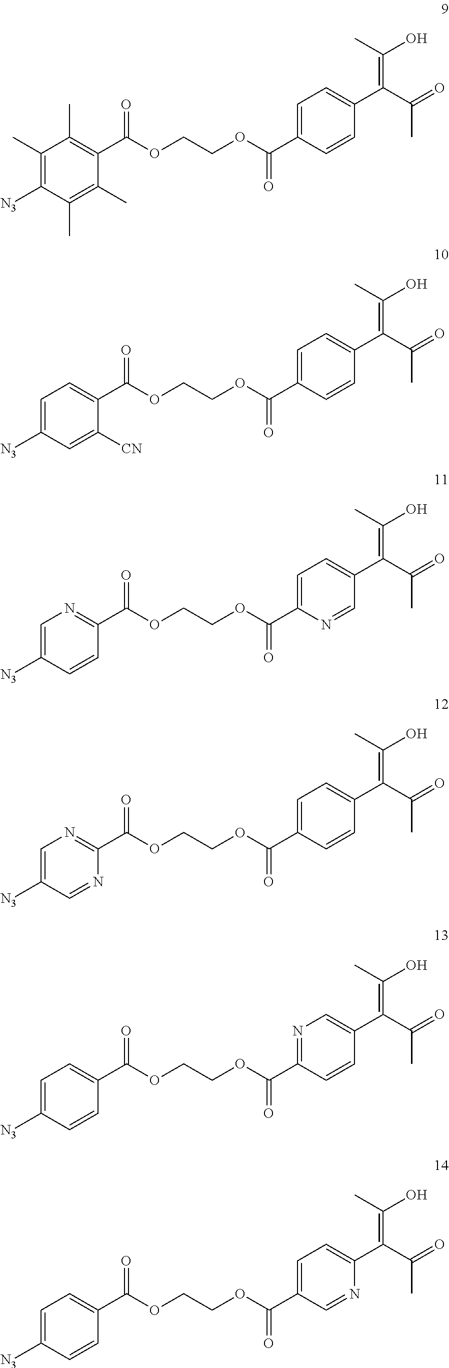 [SMILES: CC(=O)/C(=C(/C)O)c1ccc(C(=O)OCCOC(=O)c2c(C)c(C)c(N=[N+]=[N-])c(C)c2C)cc1 ; CC(=O)/C(=C(/C)O)c1ccc(C(=O)OCCOC(=O)c2ccc(N=[N+]=[N-])cc2C#N)cc1 ; CC(=O)/C(=C(/C)O)c1ccc(C(=O)OCCOC(=O)c2ccc(N=[N+]=[N-])cn2)nc1 ; CC(=O)/C(=C(/C)O)c1ccc(C(=O)OCCOC(=O)c2ncc(N=[N+]=[N-])cn2)cc1 ; CC(=O)/C(=C(/C)O)c1ccc(C(=O)OCCOC(=O)c2ccc(N=[N+]=[N-])cc2)nc1 ; CC(=O)/C(=C(/C)O)c1ccc(C(=O)OCCOC(=O)c2ccc(N=[N+]=[N-])cc2)cn1]

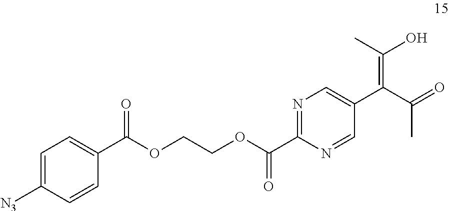 [SMILES: CC(=O)/C(=C(/C)O)c1cnc(C(=O)OCCOC(=O)c2ccc(N=[N+]=[N-])cc2)nc1]

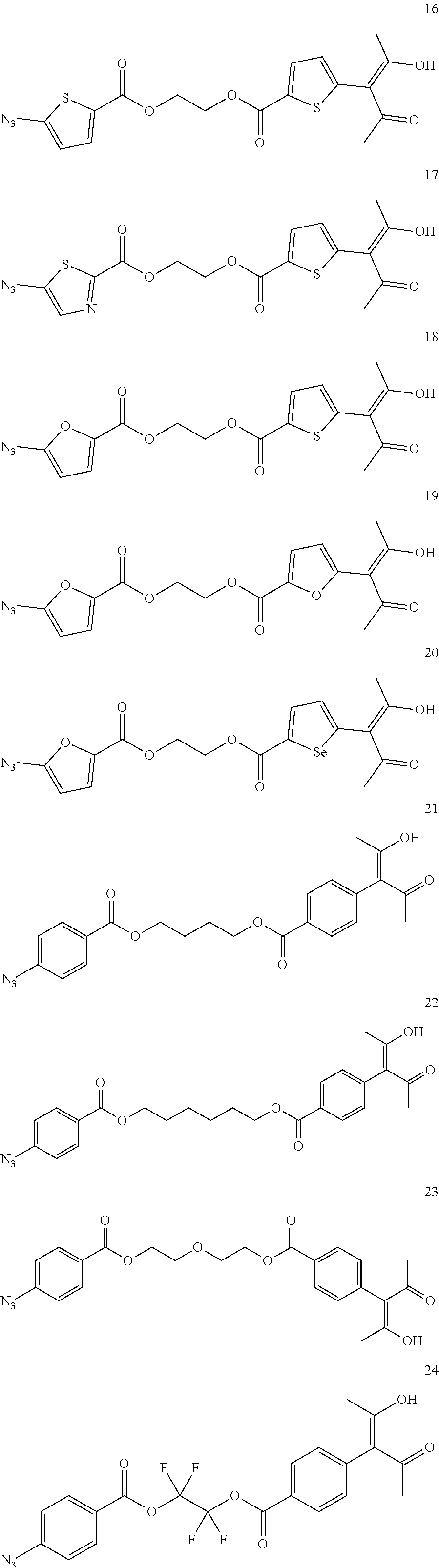

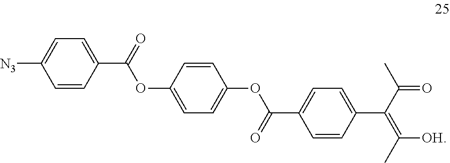 [SMILES: CC(=O)/C(=C(/C)O)c1ccc(C(=O)Oc2ccc(OC(=O)c3ccc(N=[N+]=[N-])cc3)cc2)cc1]

18. An organic semiconductor thin film comprising:
metal oxide particles; and
an organic compound represented by Formula 1 or an organic ligand represented by Formula 1A or Formula 1B: 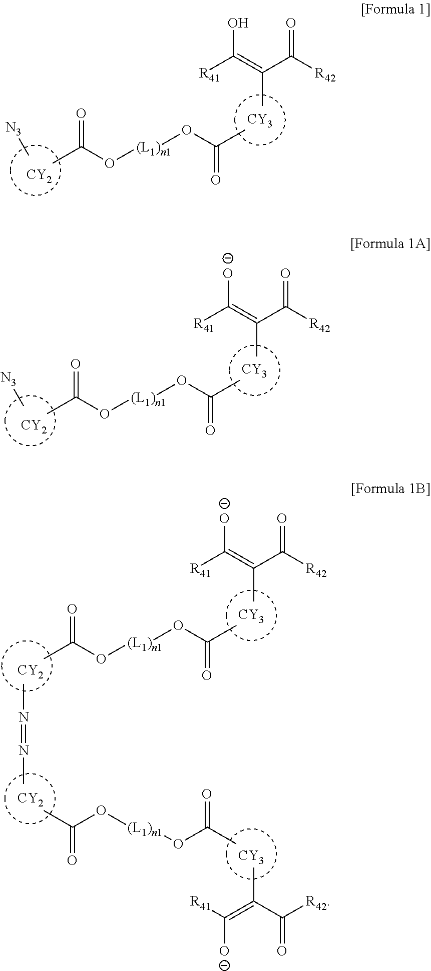
wherein in Formulae 1, 1A, and 1B,
ring CY₂ is a C₃-C₆₀ carbocyclic group or a C₁-C₆₀ heterocyclic group, each unsubstituted or substituted with at least one R₂,
ring CY₃ is a C₃-C₆₀ carbocyclic group or a C₁-C₆₀ heterocyclic group, each unsubstituted or substituted with at least one R₃,
L₁ is:
a single bond;
—O—*′; *—S—*′; or *—Se—*′; or
a C₁-C₆₀ alkylene group, a C₂-C₆₀ alkenylene group, a C₂-C₆₀ alkynylene group, a C₆-C₆₀ arylene group, or a C₁-C₆₀ heteroarylene group, each unsubstituted or substituted with at least one R₁,
* and *′ each indicate a binding site to a neighboring atom,
n1 is an integer from 0 to 10,
when n1 is 0, L₁ is a single bond,
when n1 is 2 or more, a plurality of L₁ are identical to or different from each other,
R₁, R₂, and R₃ are each independently:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or a combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
R₄₁ and R₄₂ are each independently:
hydrogen, deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁)(Q₁₂), —B(Q₁)(Q₁₂), —C(═O)(Q₁₁), —S(═O)₂(Q₁₁), —P(═O)(Q₁₁)(Q₁₂), or a combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or a combination thereof.

19. The organic semiconductor thin film of claim 18, wherein the metal oxide particles form at least one coordinate bond with the organic compound or the organic ligand.

20. The organic semiconductor thin film of claim 18, wherein
the organic semiconductor thin film comprises two or more organic compounds each independently represented by Formula 1, and
an azide group of one of the two or more organic compounds forms at least one cross-link with an azide group of another of the two or more organic compounds.
